(19) United States
(12) Patent Application Publication
(10) Pub. No.: US 2026/0354372 A1
(43) Pub. Date: Nov. 12, 2026

(54) VALVE ASSEMBLY AND VENTILATOR WITH SPOOLS FOR PROPORTIONAL FLUID FLOW CONTROL

(71) Applicant: TransMedics, Inc., Andover, MA (US)

(72) Inventors: Darren H. FREED, Edmonton (CA); Katie Gwendlyn CAMERON, Edmonton (CA); Steven Cruz ANTUNES, Edmonton (CA)

(21) Appl. No.: 19/117,746

(22) PCT Filed: Oct. 3, 2023
(86) PCT No.: PCT/US2023/075808

§ 371 Date: Apr. 2, 2025

Related U.S. Application Data
(60) Provisional application No. 63/412,664, filed on Oct. 3, 2022.

(51) Int. Cl. A01N 1/143

(52) U.S. Cl. CPC A01N 1/143

(57) ABSTRACT

A valve assembly includes a conduit comprising a first end and a second end and two control valves. Each valve comprises three ports and a spool for selectively adjusting a proportion of fluid flows through two of the ports. First ports of the valves are for connection With intake or output port of a pump respectively. Second ports of the valves are connected to the second end of the conduit for withdrawing fluid from the conduit or supplying fluid to the conduit respectively. The valve may be included in a ventilator to control the application of variable pressure to exterior surface of a lung in a sealed chamber to cause the lung to breathe.

### CROSS-REFERENCE TO RELATED APPLICATION

This application claims the priority and benefit of U.S. Provisional Patent Application Ser. No. 63/412,664, filed Oct. 3, 2022, the entire content of which is incorporated herein by reference.

### INCORPORATION BY REFERENCE

Any patent, patent publication, journal publication, or other document cited herein is expressly incorporated herein by reference in its entirety.

### TECHNICAL FIELD

This disclosure relates generally to valve assemblies and ventilators, and particularly to valve assemblies and ventilators for ex vivo ventilation of excised lungs.

### BACKGROUND

To use excised donor lungs for transplantation, the excised lungs may need to be perfused and ventilated ex vivo to restore or preserve their functionalities before the transplant procedure can be performed, or to assess or evaluate their quality or suitability for transplantation.

Various ventilation technologies have been proposed, including negative pressure ventilation (NPV) methods and related devices. In an NPV method, lungs may be ventilated utilizing an external negative pressure (i.e., below atmospheric pressure) around the lungs to allow the lungs to naturally fill with air (or a gas) that is at or near atmospheric pressure. For example, a gas such as air may be supplied at a positive pressure (above atmospheric pressure) to the airway of the lungs and a negative external pressure may be maintained around the lungs.

It is, however, desirable to provide improved devices and systems for implementing NPV, and other ventilation methods or techniques.

### SUMMARY

In an aspect of the disclosure, there is provided a valve assembly comprising a first conduit comprising a first end and a second end; and a first control valve and a second control valve, each control valve comprising a first port, a second port, and a third port, and each control valve further comprising a spool configured to selectively adjust a proportion of fluid flows through the second port and the third port of the respective control valve, wherein the first port of the first valve is configured to connect to an intake port of a pump, the second port of the first control valve is configured to connect to the second end of the first conduit for withdrawing fluid from the first conduit, the first port of the second control valve is configured to connect to an output port of the pump, and the second port of the second control valve is configured to connect to the second end of the first conduit for supplying fluid to the first conduit.

In various embodiments, the valve assembly described in the preceding paragraph may include one or any combination of the following features. The control valve may comprise an actuator for actuating the spool of the respective control valve. The actuator may comprise a servomotor. The spool of at least one each control valve of the first control valve and a second control valve is a sliding spool, and the actuator of the at least one control valve is a linear actuator. The spool of at least one control valve of the first control valve and a second control valve is a rotary spool, and the actuator of the at least one control valve is a rotary actuator. The linear actuator of the at least one control valve comprises a drive shaft coupled to the sliding spool of the respective control valve for positioning the sliding spool. The linear actuator of the at least one control valve comprises a proximity sensor for determining a position of the drive shaft of the respective control valve. The actuator of the at least one control valve comprises a controller for controlling movement of the drive shaft of the respective control valve based on an output signal from the proximity sensor of the respective control valve. The drive shaft of the linear actuator of the at least one control valve and the sliding spool of the respective control valve are axially aligned along an axis of the drive shaft. The driveshaft of the linear actuator of the at least one control valve comprises a guide rod configured to maintain the axial alignment of the drive shaft of the respective control valve. The linear actuator of the at least one control valve comprises a connector coupled to the drive shaft and the sliding spool of the respective control valve. The drive shaft of the linear actuator of the at least one control valve and the sliding spool of the respective control valve are vertically stacked. The at least one control valve comprises a housing including opposite ends and a cylindrical bore extending between the opposite ends, the first port of the respective control valve located at a first side of the bore and the second port of the respective control valve and the third port of the respective control valve located at a second side of the bore, and wherein the spool of the respective control valve is slidable in the bore and comprises a laterally extending conduit including a first opening facing the first side of the bore and a second opening facing the second side of the bore, the first opening sized and positioned to allow fluid communication with the first port, and the second opening sized and positioned to selectively allow fluid communication with the second port and the third port by sliding the spool in the bore. The valve assembly may comprise a three-way connection at the second end of the first conduit, for connecting the second end of the first conduit to the second port of the first valve and the second port of the second valve respectively. The three-way connection may comprise a three-way valve, a T-junction, or a Y-junction.

In another aspect, there is provided a ventilator comprising a sealed chamber for housing a lung in the sealed chamber comprising a pressure port; and a first fluid system for applying a variable first pressure to an exterior surface of the lung in the sealed chamber through the pressure port to cause the lung to breathe. The first fluid system comprises a first pump comprising an intake port and an output port. A valve assembly as described herein is connected to the intake port and the output port of the first pump. The first end of the first conduit of the valve assembly is sealingly coupled to the pressure port of the sealed chamber.

In various embodiments, the ventilator described in the preceding paragraph may include one or any combination of the following features. The ventilator may further comprise a second fluid system for applying and maintaining a second pressure in an airway of the lung, wherein the second fluid system may comprise a second conduit having a first end and a second end, the second end of the second conduit being connectable to a trachea of the lung through the sealed chamber. The second fluid system may further comprise a second pump for supplying a pressured fluid to the second conduit and maintaining the second pressure in the second conduit. The ventilator may further comprise a control system configured and connected for controlling the first fluid system and the second fluid system to cause the lung to breathe. The control system may comprise sensors for sensing pressures and fluid flow rates in the first conduit and the second conduit, and a processor for processing the sensed pressures and fluid flow rates and determining a pumping speed of each pump and the proportions of fluid flows through the second port and the third port of the respective control valves. The ventilator may further comprise a third conduit connecting the third port of the second control valve of the first fluid system to the second conduit of the second fluid system, and a third control valve in the third conduit for regulating fluid flow from the first control valve to the second conduit through the third conduit. Each of the first pump and the second pump may comprise a blower. The ventilator may comprise a fluid filter in each one of the first and second conduits. The ventilator may comprise a fluid filter coupled to the third port of at least one of the first control valve and the second control valve. The ventilator may comprise a fluid filter coupled to an input port of the second pump.

Other aspects, features, and embodiments of the present disclosure will become apparent to those of ordinary skill in the art upon review of the following description of specific embodiments in conjunction with the accompanying figures.

### BRIEF DESCRIPTION OF THE DRAWINGS

In the figures, which illustrate example embodiments:

FIG. 1 is a schematic block diagram of an apparatus for ventilating lungs, according to an example embodiment of the present disclosure;

FIG. 2A. is a schematic diagram of a valve assembly suitable for use in the apparatus of FIG. 1, according to an example embodiment of the present disclosure;

FIG. 2B is a schematic diagram of a valve suitable for use in the valve assembly of FIG. 2A or in the apparatus of FIG. 1, according to an example embodiment of the present disclosure;

FIG. 3 is a schematic block diagram of an example implementation of the apparatus of FIG. 1;

FIG. 4 is a schematic diagram of the apparatus of FIG. 3, showing air flow during inhalation;

FIG. 5 is a schematic diagram of the apparatus of FIG. 3, showing air flow during exhalation;

FIG. 6A is a more detailed schematic diagram of the valve of FIG. 2B;

FIG. 6B is a schematic cross-sectional view of the valve body of an example embodiment of the valve of FIG. 6A;

FIG. 6C is a schematic cross-sectional view of the valve body of another example embodiment of the valve of FIG. 6A;

FIG. 6D is a schematic cross-sectional view of the valve body of a further example embodiment of the valve of FIG. 6A;

FIG. 7A is a front perspective view of a three-way proportional valve, according to an example embodiment of the present disclosure;

FIG. 7B is a rear perspective view of the valve the valve of FIG. 7A;

FIG. 8A is an exploded front view of the valve of FIG. 7A;

FIG. 8B is an exploded rear view of the valve of FIG. 7A;

FIGS. 9A, 9B, and 9C are top cross-sectional views of the valve of FIG. 7A, along the axial line, with the spool in different positions;

FIGS. 10A, 10B, 10C, 10D, 10E, and 10F are right- or left-side cross-sectional views of the valve of FIG. 7A along the axial line, with the spool in different positions;

FIG. 11A is a front perspective view of another three-way proportional valve, according to an embodiment of the present disclosure;

FIG. 11B is a front exploded view of the valve of FIG. 12A;

FIG. 12A is a top cross-sectional view of the valve of FIG. 11A, along the axial line;

FIG. 12B is a right-side cross-sectional view of the valve of FIG. 11A along the axial line;

FIG. 13A is a left-side plane view of the spool and actuator of the valve of FIG. 11A in isolation;

FIG. 13B is a rear view of the spool and actuator of the valve of FIG. 11A in isolation;

FIG. 14 is a top perspective view of another three-way proportional valve, according to a further embodiment of the present disclosure;

FIG. 15 is an exploded view of the valve of FIG. 14;

FIG. 16A is a top cross-sectional view of the valve of FIG. 14 along the axial line;

FIG. 16B is a left-side cross-sectional view of the valve of FIG. 14 along the axial line;

FIG. 16C is a right-side cross-sectional view of the valve of FIG. 14 along the axial line;

FIG. 16D is an enlarged view of part of the valve of FIG. 15;

FIG. 17 is a top perspective view of another three-way proportional valve, according to another embodiment of the present disclosure;

FIG. 18A is an exploded view of the valve of FIG. 17;

FIG. 18B is a cross-sectional view of the valve of FIG. 17;

FIG. 19A is a top cross-sectional view of the valve of FIG. 17 along the axial line;

FIG. 19B is a right-side cross-sectional view of the valve of FIG. 17 along the axial line;

FIG. 20 is a front perspective view of another three-way proportional valve, according to a further embodiment of the present disclosure;

FIG. 21 is a front exploded view of the valve of FIG. 20;

FIGS. 22A, 22B, and 22C are top cross-sectional views of the valve of FIG. 20 along line AA-AA, with the spool in different positions;

FIG. 23 is top view of the valve of FIG. 20; and

FIG. 24 is a perspective view of the spool of the valve of FIG. 20 in isolation;

FIG. 25 is a line graph illustrating intrathoracic pressure (ITP) and airway pressure over time for a pair of ventilated lungs according to an embodiment of the present disclosure;

FIG. 26 is a schematic diagram illustrating the control logic used to control the valves and pumps of the apparatus of FIG. 3; and

FIGS. 27A and 27B are flow charts illustrating algorithms executed by the computer device if FIG. 27 for controlling the apparatus of FIG. 27.

### DETAILED DESCRIPTION

It has been recognized that, when excised lungs are ventilated ex vivo by varying a pressure around the lungs to cause the lungs to breathe, the lungs may still benefit from application of a regulated positive pressure into the airway to prevent alveolar collapse during expiration. For example, application of a continuous positive airway pressure (CPAP) combined with oscillation of a pressure around the exterior of the lungs to drive breathing may allow the transpulmonary pressure (TPP) gradient in the lungs to be conveniently regulated to allow for effective recruitment of lung parenchymal alveolar segments, while reducing, minimizing or even preventing over distension of recruited segments.

It has been further recognized that for practical application of the contemplated ventilation strategies, it is desirable to provide a portable ventilation system with a relatively small footprint that is easy to maintain and operate but still reliable. For example, it is desirable to provide compact and reliable fluid and pressure systems for applying and controlling the pressures applied to the airway and exterior surfaces of the lungs. In particular, it has been recognized that a reliable and compact valve assembly would be desirable for supplying and controlling the negative pressure applied to the exterior of the lung.

Accordingly, an embodiment of the present disclosure relates to an apparatus for ventilating excised lungs, in particular a valve assembly for use in a ventilation system. The valve assembly may include conduits and valves configured and connected to conveniently control and regulate the pressure applied to the exterior of the lung with a single pump, such as a centrifugal blower or regenerative blower, and a relatively simple mechanism for directing and proportioning the fluid flow in the conduits. Specifically, a valve used in the assembly may be a control valve including three or more input or output ports. A spool is used to control the fluid flow amongst the ports. Specifically, the spool is configured to selectively adjust a proportion of fluid flowing through the different ports. One or more first valves are used to control the fluid input to the intake port of the pump. One or more second valves are used to control the fluid flow from the output port of the pump. The valves are also connected to a conduit for selectively supplying or withdrawing fluids to the conduit. The spool may be actuated with a motor. The spool may be a sliding spool and actuated by a linear actuator, or a rotating spool actuated by a rotary motor. Conveniently, such an assembly can be compact and reliable, as will be further described below.

Other embodiments relate to systems and methods for ex vivo ventilation of lungs.

In an example method, a ventilation gas is supplied to an airway (e.g., the trachea or a bronchus) of a lung and a pressure is applied to an external surface around the lung. The external pressure may be varied (e.g., cycled) between a lower level and a higher level to cause the lung to breathe, while the pressure of the ventilation gas supplied to the airway may be regulated to maintain a continuous positive airway pressure in the airway of the lung. In some applications, the airway pressure may be constant or continuously positive over a period of time during ventilation. Typically, the external pressure may be cyclically varied between the two different pressure levels. The levels may be maintained substantially constant over a period of time, or one or both of the lower and higher levels may be adjusted during ventilation. At least one of the two levels may be below 1 atm and a vacuum is formed around the lung when the applied external pressure level is below 1 atm. The ventilation gas may be filtered with a microbe filter and a humidity-moisture-exchanger (HME) filter before being supplied into the lung. The lung may be placed in a sealed chamber, and a pressure is formed in the chamber around the lung.

Embodiments of the apparatus described herein may be conveniently used for negative pressure ventilation in an ex vivo lung perfusion (EVLP) process or system, or ex situ lung perfusion (ESLP) system. Application of positive pressure into the airway of the lung, when combined with such negative pressure ventilation, allows a higher TPP to be achieved without applying excessively negative pressure to the exterior of the lung.

Embodiments disclosed herein may also allow for recovery of atelectatic alveoli, thereby facilitating extended EVLP or ESLP. It is further convenient to use at least some of the embodiments described herein to measure and obtain functional attributes of the ventilated lungs ex vivo.

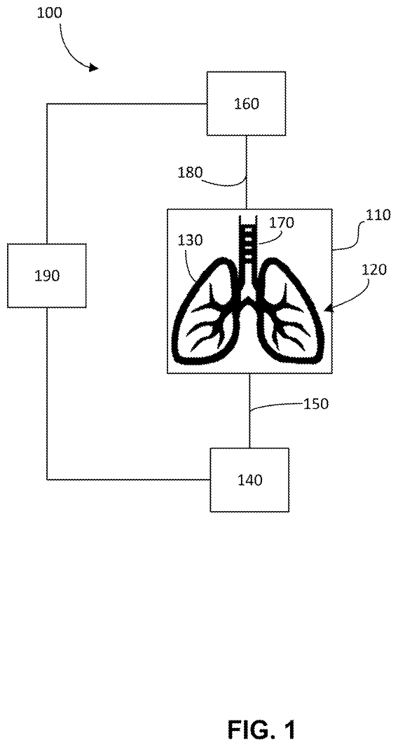

An example apparatus 100 for ventilating excised lungs according to an embodiment is schematically illustrated in FIG. 1. As depicted, the apparatus 100 includes a container 110 comprising a sealable chamber 120 for housing a lung 130. Within the container 110, the lung 130 may be supported on a flexible porous surface, such as a silicone or plastic net, or the lung may be rendered buoyant through placement on a fluid surface covered with a soft plastic membrane (not shown). Alternatively, the lung may be supported on a semi-rigid plastic form that resembles the shape of the posterior chest such that the lungs lie in an anatomically familiar position (not shown). Alternatively, the lung may be placed on a pad formed from a material resilient enough to cushion the organ from mechanical vibrations and shocks during transport. In an embodiment, the pad assembly is formed from silicone, which is biocompatible, impervious to liquids, capable of surviving sterilization processes (ETO, etc.). For clarity, it is noted that the term “a lung” can refer to a single lung, multiple lungs, or a portion of a single lung or lungs. Two lungs attached to the same trachea are sometimes collectively referred to as “a lung” or “the lung” herein.

Apparatus 100 may include a first fluid system 140 connected to chamber 120 by first conduit 150 for applying a variable first pressure to the exterior surface of the lung 130. The pressure in chamber 120 can be positive or negative at selected times. As used herein, a positive pressure refers to a pressure that is higher than the atmospheric pressure in the immediate environment of the lung and the ventilation device, unless otherwise specified expressly. A negative pressure refers to a pressure that is lower than the atmospheric pressure. That is, a positive pressure as used herein refers to a positive gauge pressure, and a negative pressure refers to a negative gauge pressure.

Apparatus 100 may further include a second fluid system 160 for applying and maintaining a second pressure, which is applied to the airway 170 of lung 130 and may be constant. Second fluid system 160 is connected by second conduit 180, which extends through the wall of the container 110 and connects the second fluid system 160 to an airway 170 of the lung 130, for supplying the second pressure to the airway of the lung. The second conduit 180 is sealed from pressure communication with the inner space in the chamber 120. The second pressure may be applied using a ventilation gas such as air or any suitable gas mixture that contains oxygen. The second fluid system 160 may include an output port of an air pump or a motor-driven turbine or other air supply mechanism (not shown in FIG. 1) for supplying air to the lung at a positive pressure. The operation speed of the air pump or turbine may be controlled to regulate airway pressure in the lung. The airway pressure may be alternatively or additionally controlled or adjusted using a valve (not shown) in the second fluid system 160.

A control system 190 may be coupled to the first fluid system 140 and to the second fluid system 160. As will be further described in more detail below, the control system 190 may be in communication with control pressure sensors, flow sensors, flow-regulating valves, to vary the pressure in chamber 120 between a lower vacuum level and a higher vacuum level to cause the lung 130 to breathe, and to regulate the pressure of the ventilation gas supplied by the second fluid system 160 to maintain a continuously positive airway pressure in the airway 170 of the lung 130.

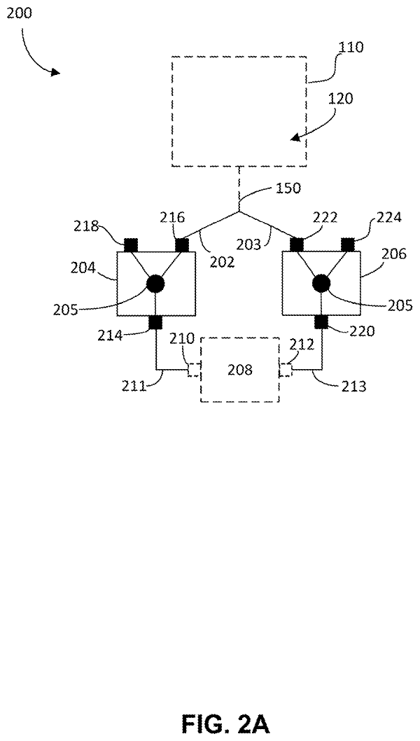

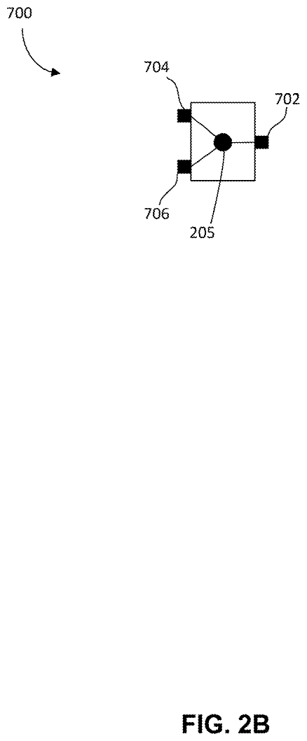

With reference to FIG. 2A, a valve assembly 200 suitable for use in fluid system 140 is shown. Valve assembly 200 is configured to supply and remove fluid to or from chamber 120 through conduit 150 with a pump 208, thus applying a variable pressure to the exterior surface of lung 130. The fluid may be air or a suitable gas supplied from a gas source (not shown). In an embodiment, the fluid is air from the atmosphere. Valve assembly 200 includes a conduit 202 for connection with conduit 150 for removing the fluid from conduit 150 and a conduit 203 for supplying the fluid to conduit 150. Valve assembly 200 also includes two three-way control valves 204, 206 with ports 216, 222 for connecting with conduit 150 and ports 214 and 220 for connecting with intake port 210 and output port 212 of pump 208 respectively. Valve 204 also has an air intake port 218, and valve 206 has an exhaust port 224.

In particular, port 214 of valve 204 is an output port and is connected to the intake port 210 of pump 208 by conduit 211. Port 216 of valve 204 is an input port and is connected to conduit 202 for removing fluid from chamber 120 through conduit 150. Port 220 of valve 206 is an input port and is connected to the output port 212 of pump 208 by conduit 213. Port 222 is an output port and is connected to conduit 203 for supplying fluid to chamber 120 through conduit 150.

Each of valves 204, 206 may have construction as shown in FIG. 2B, which illustrates a three-way control valve 700. Valve 700 has a port 702 positioned on a first side of the valve 700 and is in fluid communication with two ports 704 and 706 positioned on a second side of the valve 700, which as depicted in FIG. 2A is opposite to the first side. A control part 205 is provided in valve 700 to proportion the flow of fluid between the two ports 704 and 706, as will be further described below. Fluid flow through valve 700 may be in the direction from port 702 to ports 704 and 706, or in the reverse direction.

When valve 700 is used and orientated as valve 204 in FIG. 2A, control part 205 controls the proportion of fluids entering the valve 204 through port 216 or 218. The fluid removed from chamber 120 via conduits 150, 202 can enter valve 204 through port 216. New fluid, such as air or oxygen enriched air, can enter valve 204 through intake port 218, thus added to the fluid flow.

When valve 700 is used and orientated as valve 206 in FIG. 2A, control part 205 controls the proportion of fluids exiting through ports 222 and 224. By allowing fluid to exit through port 222, fluid is added to chamber 120 via conduits 203, 150. By allowing fluid to exit through exhaust port 224, fluid can be removed from the fluid flow. In the manner described above, fluid can be supplied and removed to/from chamber 120 by pump 208 without the need to vary the pump speed or the use of multiple pumps.

When pump 208 and conduit 150 are connected to the valve assembly 200 as described above and shown in FIG. 2A, the pump 208 may be used to control and regulate the pressure in chamber 120 as will be further described below. Valve assembly 200 and pump 208 thus form part of the fluid system 140. Fluid system 140 may include other components such as sensors, filters, processors and additional pumps.

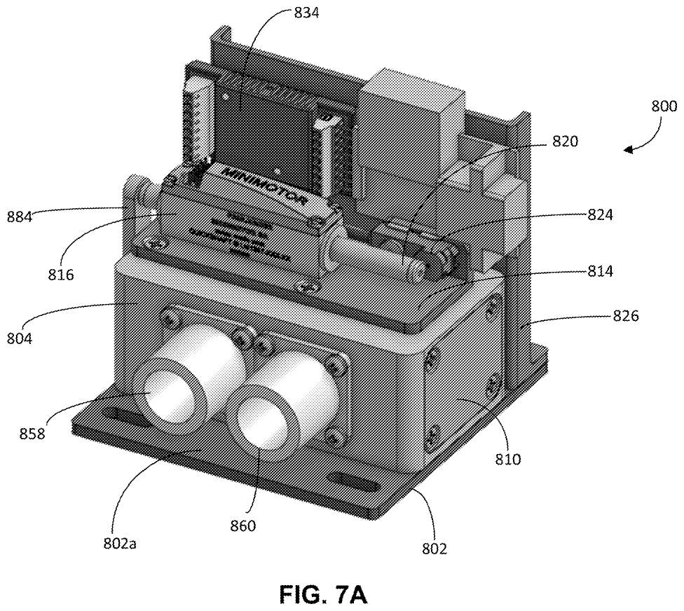

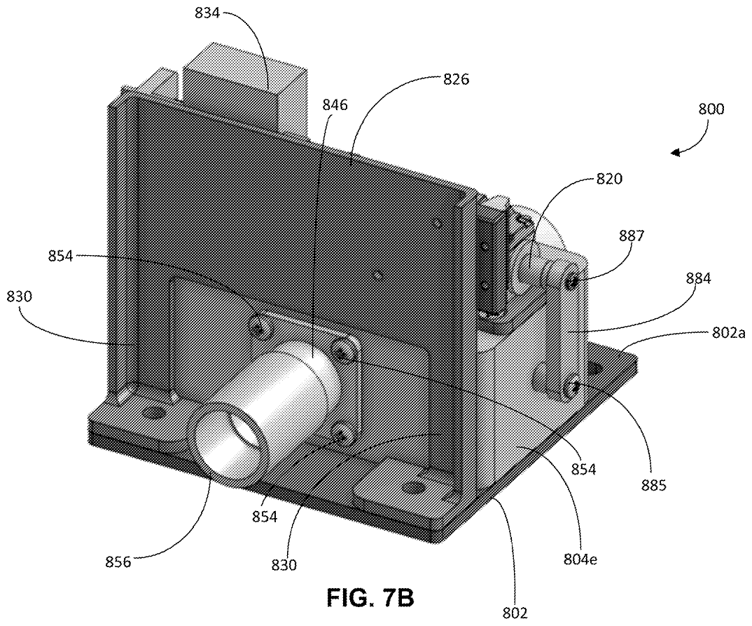

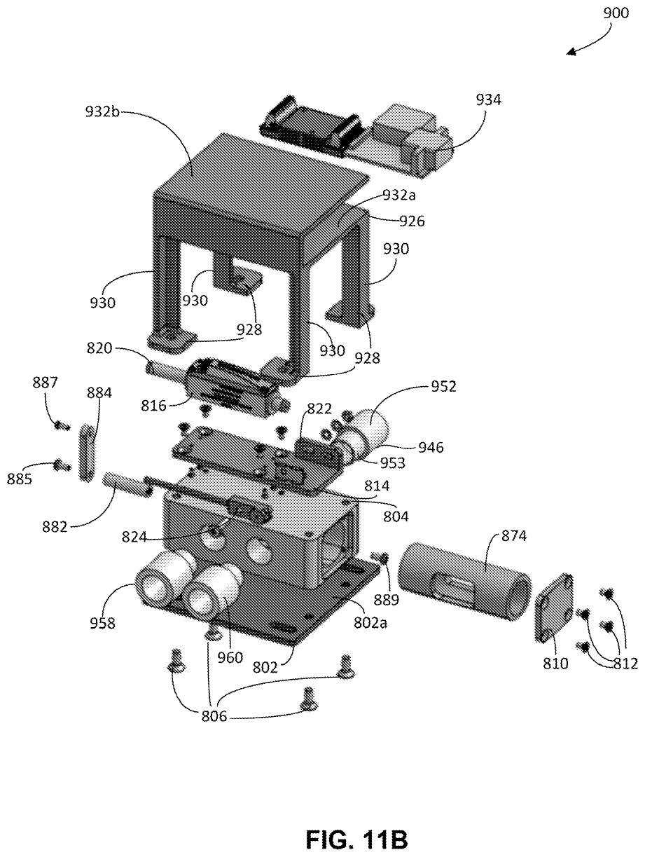

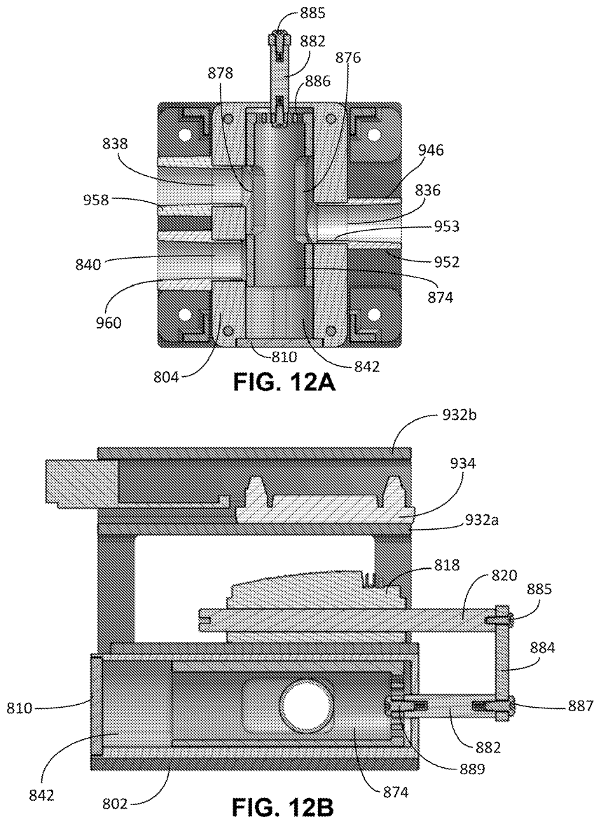

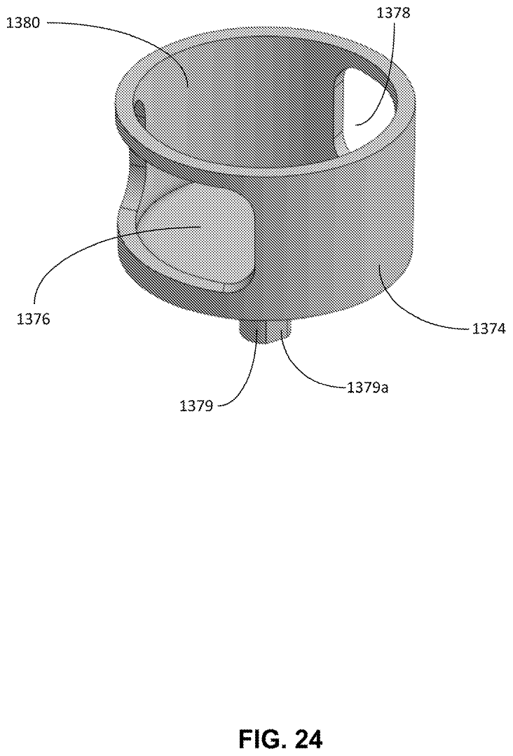

Possible constructions and example embodiments of the valve 700 will be described below with reference to FIGS. 7 to 24.

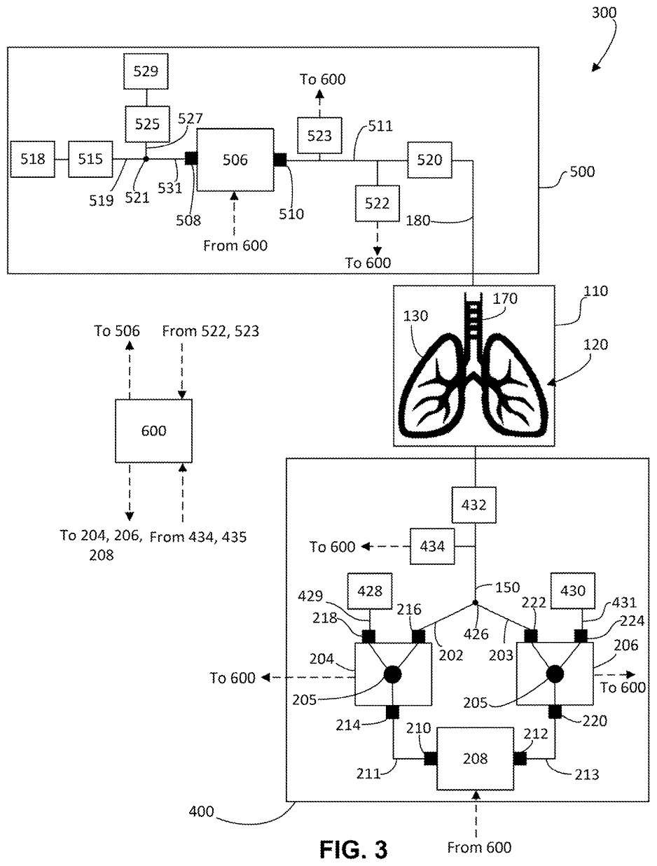

FIG. 3 illustrates an apparatus 300, which is an example embodiment of the apparatus 100 shown in FIG. 1, and in which the use of the valve assembly 200 is illustrated. Apparatus 300 includes a fluid system 400, an example embodiment of the first fluid system 140 described above, with the valve assembly 200 as shown in FIG. 2A. Fluid system 400 further includes a three-way connection 426 connecting conduit 150 with ports 216 and 222 via conduits 202, 203 for either removing fluid from or adding fluid into conduit 150. Three-way connection 426 may include a three-way valve, or a T-junction or a Y-junction, or the like.

Fluid system 400 also includes a fluid filter 428 mounted at or connected to port 218 of valve 204 via conduit 429 and a fluid filter 430 mounted at or connected to port 224 of valve 206 via conduit 431. Filters 428, 430 may be high efficiency particulate air (HEPA) filters in order to filter particulates out of the incoming air. A filter 432 may also be positioned in conduit 150 and may be a humidity and moisture exchanger (HME) filter to retain humidity in chamber 120 to avoid desiccation of the lungs 130.

Fluid system 400 may further include an integrated sensor 434, coupled to conduit 150. Sensor 434 can be configured to detect pressure, flow, humidity, gas content and the like within conduit 150. Sensor 434 may be a FS6122-250F250-100P 100-TH1 sensor made by Siargo™.

Apparatus 300 further includes a second fluid system 500, which is an example embodiment of fluid system 160 described above. Fluid system 500 is configured to apply and maintain an airway pressure in the airway 170 of the lung 130. Fluid system 500 is coupled to the airway 170 via conduit 180 and includes a pump 506 connected as illustrated. Specifically, pump 506 has an intake port 508 and output port 510. Intake port 508 receives a fluid supply from conduit 531, which may include a three-way connection 521, which may be a three-way valve, or a T-junction or a Y-junction. Fluid may be drawn from the atmosphere through conduit 519 and into conduit 531 via three-way connection 521. Conduit 519 may also include a valve 515 for controlling fluid flow from the atmosphere. Alternatively, oxygen may be supplied to conduit 531 from an oxygen supply 529 via conduit 527. Conduit 527 may also include a valve 525 for controlling oxygen flow from oxygen supply 529. Valves 515 and 525 may be 2-way proportional solenoid valves, and may be optionally controlled by control system 600. Through control of valve 525 the supply of air or a mixture of air and oxygen to conduit 531 may be adjusted.

Fluid system 500 may further include fluid filters 518, 520. Fluid filter 518 may be mounted at or connected to conduit 519 for filtering air entering pump 506. Fluid filter 520 may be positioned between conduits 511 and 180 and may be a HME filter for retaining humidity in the trachea and preventing humidity from entering the valve. This is important in order to prevent tissue desiccation of the lungs 130 and avoid bacterial contamination within fluid system 500. Filter 518 may be a HEPA filter in order to filter particulates out of the incoming air.

Any suitable microbe filters, such as HEPA filters and HME filters known to those skilled in the art may be used as a filter in an embodiment herein. There are products on the market that have both HME and HEPA properties. In this embodiment, filters 428, 430, 432, 518, 520 are combined HEPA/HME filters.

Fluid system 500 may further comprise an integrated sensor 522, coupled to conduit 511. Sensor 522 can be configured to detect pressure, flow, or humidity within second fluid system 500. An oxygen sensor 523 may also be coupled to the output port 510 of pump 506 for detecting oxygen content within second fluid system 500.

Apparatus 300 further includes a control system 600, which is an example embodiment of the control system 190. Control system 600 is in communication with valves 204, 206, pump 208, and sensor 434 of system 400, and with pump 506 and sensors 522, 523 of fluid system 500. In some embodiments, fluid system 400 and fluid system 500 may comprise separate or additional control systems or devices. Control system 600 is configured to control the operation of the pumps and valves to regulate the direction and pressure of fluid flow within apparatus 300.

Providing separate fluid systems 400, 500 with separate pumps in apparatus 300 allows each system 400 or 500 to be more compact and offers greater and simplified control of each of the respective systems. This configuration also reduces the risks associated with failure of a component in the apparatus because in case one of fluid systems 400, 500 fails, the other system may still properly function. That the separate fluid systems 400 and 500 each comprise a dedicated pump further allows for smaller, quieter and more compact pumps to be used in each system.

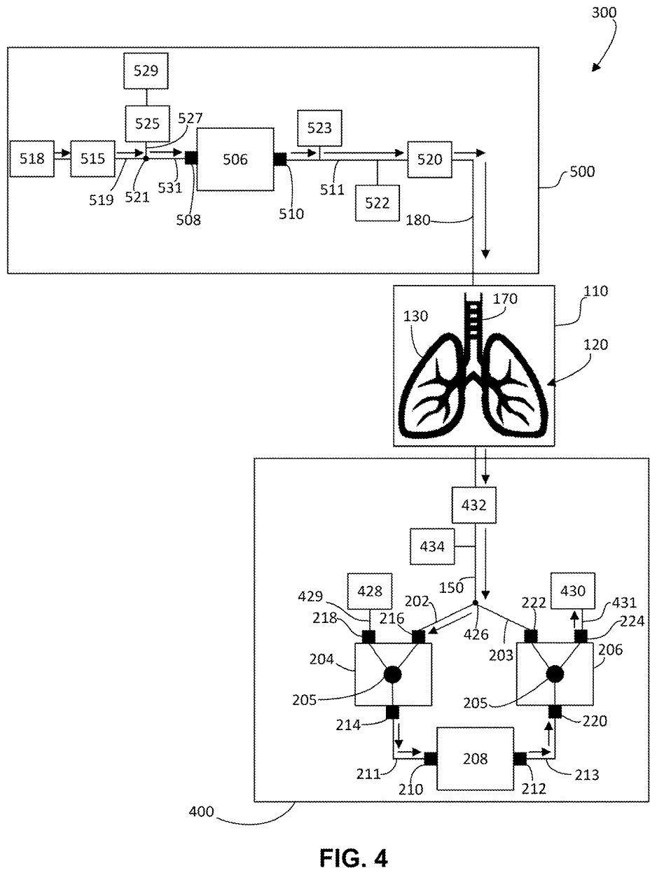

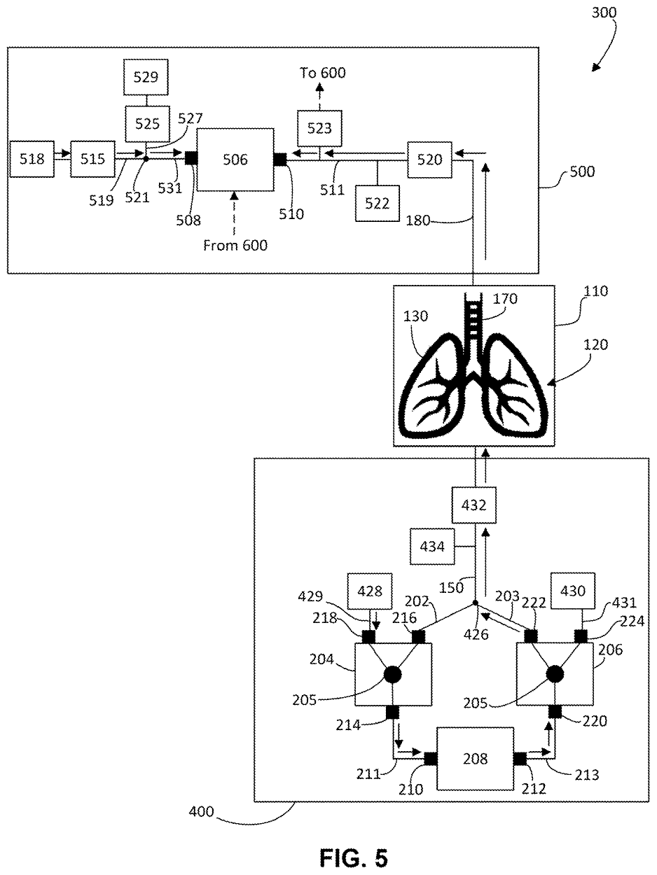

In operation, apparatus 300 may be operated to ventilate a lung ex vivo as illustrated in FIGS. 4 and 5, as described below. The lines with arrows in FIGS. 4 and 5 indicate the directions of airflow.

FIG. 4 illustrates the fluid flow in apparatus 300 during inhalation. The valves 204 and 206 are configured under control by controller 600 to allow fluid flow between port 216 and port 214 in valve 204, and between port 220 and port 224 in valve 206, but block fluid flow between port 222 and port 220 in valve 206 such that air can flow from the container 110, through filter 432, conduits 150, 202 and junction 426, into valve 204 via port 216, but not into valve 205 through port 222. For inhalation, pump 208 is operated to pump air from intake port 210 towards output port 212 at a selected pumping speed or rate. As a result, air is drawn out of the chamber 120 of container 110, and the air received at port 216 of valve 204 is directed to flow through port 214 into the intake port 210 of pump 208. The air pumped to valve 206 through output port 212 is directed by valve 206 to port 224 and then released to the atmosphere through filter 430. In this manner, the pressure inside chamber 120 of the container 110 is decreased and a negative pressure is applied to the exterior of the lungs 130. The pressure at conduit 150 may reflect closely the pressure in chamber 120, and may be measured using sensor 434. Controller 600 may be configured to monitor the pressure in container 110 (which may be measured by sensor 434) over a specified timeframe, before adjusting valve 204 to stop removing fluid from container 110 through port 216. In some embodiments, the air flow speed through conduit 150 may be controlled by the pumping speed of pump 208. In some embodiments, the air flow speed through conduit 150 may be controlled by adjusting valve 204 while the pump speed is maintained constant. For example, valve 204 may be adjusted to allow some external air to enter the air flow stream to port 214 through port 218, thus decreasing the amount of air drawn through conduits 202 and 150.

Alternatively or additionally, valve 206 may be controlled to allow a selected proportion of the air flowing through port 220 to flow through port 222, conduit 203 and junction 426 back to conduit 202, which would also decrease the air flow rate from container 110 to junction 426 through conduit 150. In some embodiments, the fluid flow rate in conduit 150 may be controlled by adjusting two or more of valves 204, 206 and pump 208.

For example, in some applications, valve 204 may be controlled by way of state control, i.e., controlling movement of the valve from the 0% (fully closed) position or state to the 100% (fully open) position or state, to control the breath state; and valve 206 may be used to regulate the pressure and inspiratory rise time (Tᵢ) in chamber 120.

During the same inhalation period, subsystem 500 provides air or an oxygen-containing gas into lungs 130 through airway 170. For example, pump 506 may be operated to pump air from intake port 508 to output port 510 at a selected speed. As a result, external (e.g. environmental) air is drawn into the intake port 508 of pump 506 through conduits 519, 531, and filter 518 and is supplied to air way 170 through output port 510, conduit 511, filter 520, and conduit 180. In this manner, a positive pressure is applied to the airway 170 of the lungs 130. In one operation mode, valve 525 may be closed. In a different operation mode, valve 525 may be open so that the oxygen gas in oxygen supply source 529 may be pumped through conduit 527 and mixed with air in conduit 521 and 531 at connection 521. As a result, the air supplied to airway 170 is oxygen-enriched. The proportion of oxygen added to the air flow may be regulated by adjusting valve 525 under control of the controller 600. The oxygen content or level in the air in conduit 511 may be measured using oxygen sensor 523, and valve 525 may be controlled based on the measured oxygen level by controller 600.

The pressure differential between the positive pressure applied to the airway 170 and the negative pressure applied to the exterior of the lungs 130 cause the lungs to expand and thus inhale air.

The combination of applied negative and positive pressures as described above also result in a pressure gradient from the airway 170 to the alveoli, which causes some air to flow into and through the alveoli. Some air may pass through the lungs 130 and enter into chamber 120.

FIG. 5 illustrates the air flow in the apparatus 300 during exhalation in some situations. The valves 204 and 206 are configured under control by controller 600 to allow fluid flow between port 218 and port 214 in valve 204, and between port 220 and port 222 in valve 206, but block fluid flow between port 214 and port 216 in valve 204 such that air can flow from valve 206 to the container 110, through port 222, conduits 203 and 150, junction 426, and filter 432, but not from port 216 to port 214. For exhalation, pump 208 is still operated to pump air from intake port 210 towards output port 212 at a selected pumping speed. As a result, external air is drawn into valve 204 through port 218 and filter 428, and then into pump 208 through conduit 211 and intake port 210. The air is pumped to valve 206 through output port 212 and conduit 213, and is directed by valve 206 to port 222 and supplied back to container 110 through conduits 222, 150, junction 426, and filter 432. In this manner, the pressure inside chamber 120 of the container 110 is increased, and the pressure applied to the exterior of the lungs 130 is increased. It is noted that during exhalation, it is possible that the pressure in the airway is negative, in which case, apparatus 300 may operate in a manner similar to that in the inhalation period as described above.

During the exhalation period, subsystem 500 may continue to supply air or oxygen-enriched air to the airway 170 of lungs 130 and maintain a positive air pressure in the airway 170 of the lungs 130, as in the inhalation period as described above.

In any event, during exhalation, the increased pressure outside the lungs 130 causes the pressure differential between the airway 170 and chamber 120 to decrease, and consequently causes the lungs to exhale and air to flow from the alveoli to the airway 170 and out of the lungs 130.

Through the adjustment on control part 205, valves 204 and 206 can be controlled by controller 600 to alternately either add or remove fluid from conduit 150, therefore causing the pressure inside chamber 120 to oscillate between a lower pressure level and a higher-pressure level.

Control system 600 may function in a number of different ways to control the inhalation and exhalation cycles of apparatus 300. In a first embodiment, pump 208 may be running constantly at a fixed speed throughout the inhalation and exhalation cycles and control system 600 adjusts valves 204 and 206 to operate proportionally through adjustment of control part 205 in order to proportion the airflow appropriately. That is, airflow may be directed from either of the ports 216 and 218 to port 214 of valve 204 or the proportion of fluids entering the valve 204 through ports 216 and 218 may be controlled. In a similar manner, airflow may be directed from port 220 of valve 206 to either of ports 222 and 224, or may be proportioned between ports 222 and 224.

In another embodiment, control system 600 regulates and adjusts the speed of pump 208 whilst adjusting valves 204 and 206 as non-proportional three-way valves between a number of setpoint positions. As will be outlined in more detail below, control system 600 may operate to regulate and adjust the speed at which a valve moves between the setpoint positions in order to control the timing between stages of a respiratory profile. That is, airflow may be directed from either of ports 216 and 218 to port 214 of valve 204 but is not proportioned from ports 216 and 218 to port 214. In a similar manner, airflow may be directed from port 220 of valve 206 to either of ports 222 and 224, but is not proportioned between ports 222 and 224.

As can be appreciated, the speed at which the valves move between different set positions or open/close states can have significant effects on the breath timing control. Therefore, in embodiments disclosed herein, system 300 may be configured to vary the speed of movement of each valve between different valve positions depending on the timing setpoints, which may be set by an operator or user. Such speed control may be conveniently achieved with example valve assemblies described herein.

In another embodiment, control system 600 may regulate and adjust the speed of pump 208 whilst also adjusting valves 204 and 206 to operate proportionally through adjustment of control part 205 in order to proportion the airflow appropriately. That is, airflow may be directed from either of the ports 216 and 218 to port 214 of valve 204 or the proportion of fluids entering the valve 204 through ports 216 and 218 may be controlled. In a similar manner, airflow may be directed from port 220 of valve 206 to either of ports 222 and 224, or may be proportioned between ports 222 and 224.

Control system 600 may also function to maintain a constant positive airway pressure (CPAP) within airway 170 though regulation and adjustment of the speed of pump 506 whilst adjusting valves 515 and 525 between a number of fixed positions. In an embodiment, pump 506 may be running constantly at a fixed speed and control system 600 adjusts valves 515 and 525 such that fluid flows from the atmosphere, through conduits 519, 531, 511, 180 and into airway 170. In another embodiment, control system 600 adjusts valves 515 and 525 such that oxygen from oxygen supply source may be mixed with air from conduit 519 and 531 at connection 521. The oxygen content or level in the air in conduit 511 may be measured using oxygen sensor 523, and valve 525 may be controlled based on the measured oxygen level by controller 600. In some embodiments, when the pressure in conduit 180 is controlled using a pump, such as pump 506, valve 515 may be omitted.

Controller 600 may control the pumps and valves in apparatus 300 to provide a desired or selected respiratory profile.

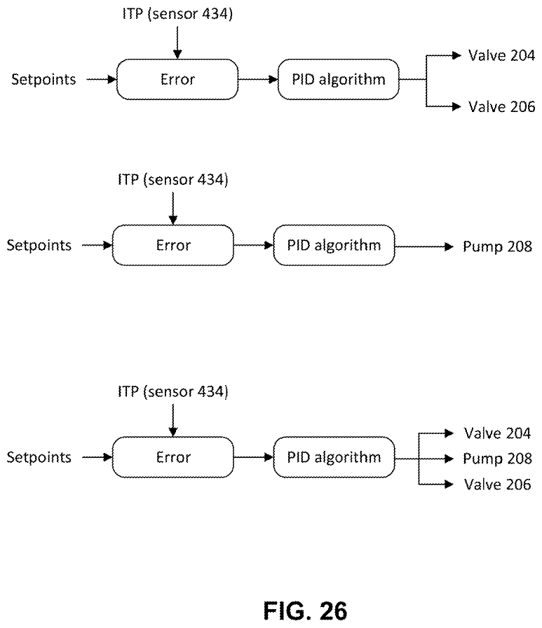

An example respiratory profile is shown in FIG. 25. In this example, the set-point for ITPᵢ (inspiratory intrathoracic pressure) was −11 cmH₂O and the set-point for ITPₑ (expiratory intrathoracic pressure) was 1 cmH₂O. ITPᵢ and ITPₑ correspond to the respective minimum and maximum pressures within container 110 as measured by sensor 434 during inspiration and expiration respectively.

Expiratory rise time (Tₑ) is the time taken for the ITP to reach ITPₑ once ITP begins to rise following inspiration. Inspiratory rise time (Tᵢ) is the time taken for the ITP to reach ITPᵢ once the ITP begins to fall following expiration.

Expiratory time (ET) is the calculated time span that the lungs are in the exhalation state, whilst inspiratory time (IT) is the calculated time span that the lungs are in the inhalation state. ET may be calculated from ((1/RR)×(E/I+E)). RR is the respiratory rate, which is the number of cycles per minute in breaths per minute (BPM). Similarly, IT may be calculated from ((1/RR)×(I/I+E)). I and E are simplified integers used in the I:E ratio and are the reduced forms of the ET and IT when in ratio. For example, if IT=4 seconds and ET=2 seconds, then I:E=4:2=2:1.

The cycle time (T_{cycle}) is the sum of ET and IT. Inspiration to expiration time ratio (“I:E”) is the ratio of IT to ET.

FIG. 25 illustrates two profiles for Tᵢ; Tᵢ(a) and Tᵢ(b). Tᵢ(a) corresponds to a short inspiratory rise time whilst Tᵢ(b) corresponds to a full inspiratory rise time, as measured by sensor 434. A short inspiratory time may be used, for example when lungs are non-compliant or require recruitment. A non-compliant lung may refer to a lung that has poor expandability (or poor pulmonary compliance). Lung recruitment refers to temporarily increasing airway pressure in order to open up collapsed alveoli. In such scenarios it may be beneficial to have a short inspiratory rise time, followed by a longer pause at ITPᵢ which may aid in improving lung compliance and recruitment.

In some embodiments ITPₑ may range from −10 cmH₂O to 10 cmH₂O and the minimum value for ITPᵢ may be −30 cmH₂O.

FIG. 25 also illustrates the constant positive airway pressure (CPAP) applied, as measured by sensor 523. In some embodiments the CPAP may be maintained at 7.5 cmH₂O. The desired pressure may be maintained through the adjustment of the speed of pump 506 by controller 600, based on the pressure measured in conduit 511 by sensor 523.

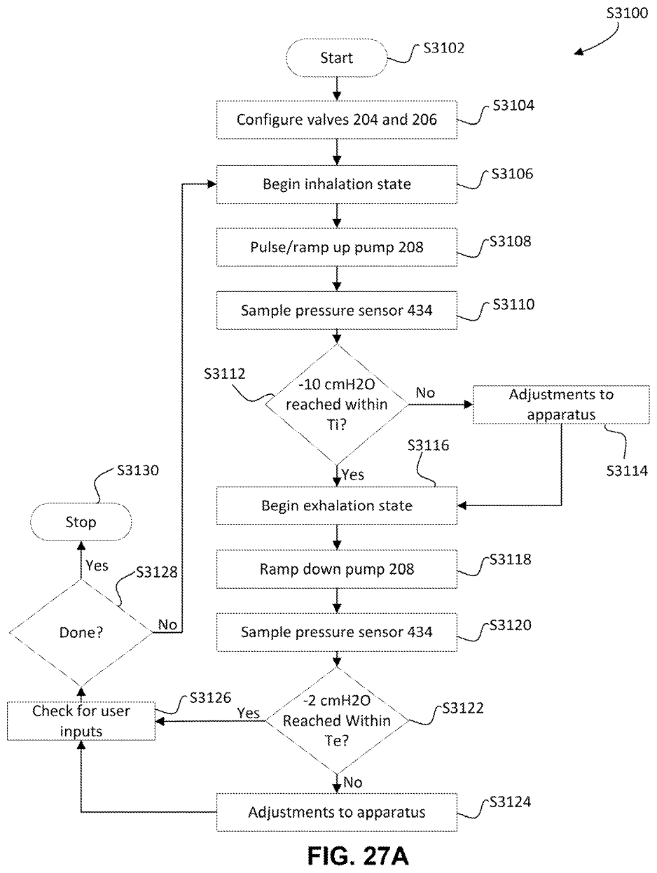

FIG. 26 illustrates the control logic for controlling valves 204 and 206, and pump 208. The control may be implemented using a proportional-integral-derivative (PID) controller, or any standard variants of PID control. As can be understood by persons skilled in the art, a PID controller continuously calculates an error value as the difference between a desired setpoint (SP) and a process variable (PV) to obtain a control output (CO). A PID controller can attempt to reach the desired setpoint over time by continually reducing the error value by adjusting the control output. A control system may include one or more PID controllers for controlling one or more variables. Multiple PID controllers may attempt to reach a multitude of desired setpoints. The set-points in an embodiment may be prestored or manually entered by a user, and may include ITPᵢ, (I-PD control), ITPₑ (I-PD control), RR, I:E, Tₑ (I-P control) and Tᵢ (I-P control).

The PID control logic shown in FIG. 26, which may be followed by control system 600, may be used to control proportional fluid flows in valves 204 and 206 by adjusting control part 205, and the speed of pump 208. In this logic, the pressure measured by sensor 434 in conduit 150 during inhalation, ITPᵢ, or during exhalation, ITPₑ (as measured by sensor 434), is compared to set-points of the desired ITPᵢ and ITPₑ. The difference between the actual values as measured by sensor 434 and the set-points are used as feedback for adjusting the valves 204 and 206 only, the speed of pump 208 only, or both valves 204 and 206 and the speed of pump 208.

During operation of the fluid system 400, the configuration of valves 204 and 206 may be dependent on the value of ITPₑ input by the user. For ITPₑ≤0, valves 204 and 206 may maintain the same configuration throughout both the inhalation and exhalation states. This configuration is achieved by adjusting control part 205 to configure valve 204 to divert air through port 214 and into pump 208, and by adjusting control part 205 to configure valve 206 to release air into the atmosphere via port 224. During each inhalation and exhalation state, the speed of pump 208 may be adjusted by control system 600 based on ITPᵢ (during inhalation) or ITPₑ (during exhalation) as measured by pressure sensor 434, to achieve the user-input target ITPᵢ and ITPₑ during inhalation and exhalation respectively.

For ITPₑ≥1, the configuration of valves 204 and 206, which is achieved through adjusting control part 205, depends on whether the system is operating in the inhalation or exhalation state. During the inhalation state, valve 204 is configured to divert air from port 216 through port 214 and into pump 208, and valve 206 is configured to release air into the atmosphere via port 224. During the exhalation state, valve 204 is configured to intake air from port 218 and into pump 208, and valve 206 is configured to divert air from port 222 and into conduit 150. During each inhalation and exhalation state, the speed of pump 208 may be adjusted by control system 600 based on ITPᵢ (during inhalation) or ITPₑ (during exhalation) as measured by pressure sensor 434, to achieve the user-input target ITPᵢ and ITPₑ during inhalation and exhalation respectively. The manner in which the speed of pump 208 is adjusted by control system 600 is dependent on the values of Tᵢ and Tₑ input by the user. For Tᵢ<2 seconds or Tₑ<2 seconds, the speed of pump 208 may momentarily pulse or ramp at the beginning of the inhalation or exhalation states, respectively, in order to accelerate the pressure in container 110 towards reaching the setpoint ITPᵢ or ITPₑ respectively.

The pulse/ramp waveform of pump 208 may be controlled by PID controllers as described above, having user inputted values of Tᵢ or Tₑ as their setpoint, where measured values of Tᵢ or Tₑ (process variables) based on which the PID controller can attempt to reach the desired setpoint over time by continually reducing the error value through adjustment of a pump speed multiplier (control output) of pump 208 whilst maintaining a constant pulse duration, which may be 0.5 seconds. The pump speed multiplier may range from 0 to 4. If the pump speed multiplier is <1, then a ramp up effect will result. The ramping from pulse to steady speed may occur over 50% of the entire inhalation or exhalation breath length.

When ITPₑ<0 cmH₂O, the speed of pump 208 will always be increased during inhalation and decreased during exhalation. For Tᵢ>2 seconds or Tₑ>2 seconds and ITPₑ≤0 cmH₂O, the speed of pump 208 changes at a calculated rate in order to achieve the pressure in container 110. If Tᵢ>2 seconds, the speed of pump 208 increases at the calculated rate in order to achieve the user-input ITPᵢ during inhalation. If Tₑ>2 seconds, the speed of pump 208 decreases at the calculated rate in order to achieve the user-input ITPₑ during exhalation.

The speed of pump 208 may be governed by equation (1),

$$b = {b}_{s} + {( \frac{t}{{CT}_{i / e}} )}^{a} ( {b}_{f} - {b}_{s} ) , \qquad ( 1 )$$

whereby b is the speed of pump 208 as a percentage of the full range (bₛ and b_{f} are the starting and final speeds of pump 208, respectively), t is the time since the start of the switch between exhalation and inhalation (or vice versa), and a and C are constants, whereby C is preferably 0.9.

Constant a may be calculated from equation (2), whereby if bs>bf, A=1.5, otherwise A=1,

$$a = \frac{{AT}_{i / e}}{10} . \qquad ( 2 )$$

For Tᵢ>2 seconds or Tₑ>2 seconds and ITPₑ>0 cmH₂O, the speed of pump 208 adjusts in the same manner mentioned above. Additionally, valves 204 and 206 are configured by control system 600 through adjusting control part 205 at a second calculated rate to achieve the pressure in container 110. If Tᵢ>2 seconds, valves 204 and 206 are adjusted at the second calculated rate in order to achieve the user-input ITPᵢ during inhalation. If Tₑ>2, valves 204 and 206 are adjusted at the second calculated rate in order to achieve the user-input ITPₑ during exhalation.

The rate of adjustment of control parts 205 of valves 204 and 206 are governed by equations (3) and (4):

$$x ( t < {t}_{0.5} ) = {x}_{0} + \frac{t}{{t}_{0.5}} ( {x}_{0.5} - {x}_{0} ) , \qquad ( 3 )$$

$$x ( t \geq {t}_{0.5} ) = {x}_{0.5} + \frac{t - {t}_{0.5}}{{T}_{i / e} - {t}_{0.5}} ( {x}_{1} - {x}_{0.5} ) . \qquad ( 4 )$$

In equations (3) and (4), x₀, x_{0.5} and x₁ represent the starting point, midpoint and final point respectively if control part 205 and t₀, t_{0.5} and t₁ represent the starting time, time at the midpoint and time at the final point respectively during adjustment of control part 205.

The parameters listed below in Table 1 are used to modify the values in equations (3) and (4) to adapt to the behavior of the physical system at different setpoints. Note that during inhalation, the subscripts s and f (relating to starting and final) indicate exhalation and inhalation, respectively; for exhalation, they are swapped. Regarding nomenclature, b refers to speed of pump 208, ITP refers to the setpoint for ITP (ITPₑ or ITPᵢ), v refers to prescribed valve position datums for valves 204 and 206, and x refers to the valve position determined within the context of the controller.

TABLE 1

| Parameters | Calculation |
|---|---|
| P | $\frac{{b}_{f}}{{b}_{s} + {b}_{f}}$ |
| M | if P < Mₘᵢₙ, M = Mₘᵢₙ; |
| | else if P > Mₘₐₓ, M = Mₘₐₓ; |
| | else, M = P |
| t_{0.5} | $- 2 M \frac{{T}_{i / e} {ITP}_{s}}{{ITP}_{f} - {ITP}_{s}}$ |
| xₛₕₒₒₜ | 2PV (v_{f} − vₛ) |
| x₀ | vₛ + xₛₕₒₒₜ |
| x₁ | v_{f} |
| x_{0.5} | x₀ + M (x₁ − x₀) |

In Table 1, the valve positions, vₛ and v_{f}, are known. The T_{i/e} and ITP_{s/f} values are the desired setpoints, which are also known. The baseline valve shoot, V, is a prescribed constant (between 0 and 1) dictating the baseline of where the valves immediately move to upon a breath state switch.

P characterizes the difference of the ramp blower speeds between the starting and final states of the breath and is used to modify the baseline valve shoot (V) to determine the contextually modified valve shoot, xₛₕₒₒₜ. This is necessary because valve shoot is dependent on the transition in speed of pump 208, and when ramping downwards, valve shooting can make the slope too severe.

M is a bounded modifier that tweaks the transition time and position, t_{0.5} and x_{0.5}, to account for the dynamic nature of the pressure equilibration of the system. It was found that M required restrictions around the halfway valve position to prevent it from overcompensating the midpoint parameters.

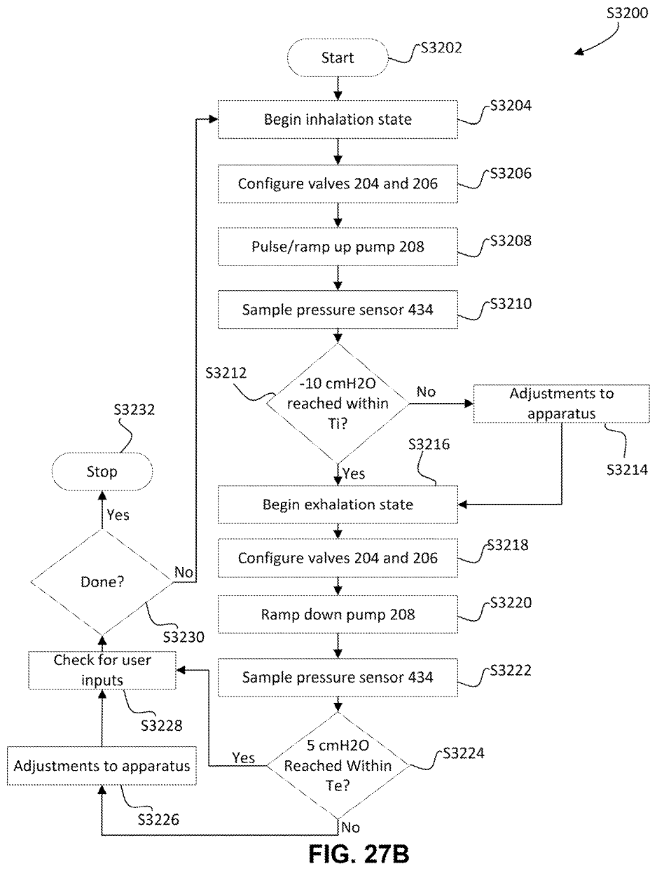

FIG. 27A is a flow chart of an algorithm S3100 that can be executed by one or more controllers, processors or computers to control an apparatus such as apparatus 300 in FIG. 3, when ITPₑ≤0. In this example, the set-points inputted by the user are ITPᵢ=−10 cmH₂O, ITPₑ=−2 cmH₂O, Tᵢ=1 second, Tₑ=3 seconds, RR=10 BPM, and I:E=1:1.

The software is initiated by a signal provided by a user or automated process at block S3102. At block S3104, valve 204 is configured by adjusting control part 205 to divert air through port 214 and into pump 208, and valve 206 is configured to release air into the atmosphere via port 224.

At block S3106, the inhalation state of the apparatus begins, and at block S3108 a signal is sent to pump 208 to pulse and ramp up to a certain speed in order increase air flow into conduit 150. At block S3110 the pressure sensor 434 is sampled, and at block S3112 the algorithm determines whether ITPᵢ has been reached within the set Tᵢ. If ITPᵢ has not been reached (as may be the case on the first ventilation cycle in particular), adjustments are made to the speed of pump 208 at block S3114 for the next ventilation cycle before proceeding to block S3116. If ITPᵢ has been reached, no adjustments are necessary and the algorithm proceeds directly to block S3116. Blocks S3112 and S3114 may include PID calculations, as discussed above with respect to FIG. 26.

The exhalation state begins at block S3116. Following the beginning of the exhalation state, at block S3118 a signal is sent to reduce the speed of pump 208 in order to reduce air flow into conduit 150 to reach the set ITPₑ within the set Tₑ. At block S3120 the pressure sensor 434 is sampled, and at block S3122 algorithm determines whether ITPₑ has been reached within the set Tₑ. If ITPₑ has not been reached (as may be the case on the first ventilation cycle in particular), adjustments are made to the speed of pump 208 at block S3124 for the next ventilation cycle before proceeding to block S3116. If ITPₑ has been reached, no adjustments are necessary and the algorithm proceeds directly to block S3126. Blocks S3122 and S3124 may include PID calculations, as discussed above with respect to FIG. 26.

At block S3126 the algorithm checks for user input (e.g., a change in set-points). At block S3128, it is determined whether the operation settings should be reconfigured. Reconfiguration may be required when a different mode of operation is desired. If the settings are not to be reconfigured, the software returns to block S3106 to repeat the inhalation state. If the settings are to be reconfigured, for example when starting a new operation mode, ventilation is stopped at block S3130.

FIG. 27B is a flow chart of another algorithm S3200 that can be executed by one or more processors S3202 to control an apparatus of the present disclosure, such as the apparatus 300 in FIG. 3, when ITPₑ≥1. In this example, the set-points inputted by the user are ITPᵢ=−10 cmH₂O, ITPₑ=5 cmH₂O, Tᵢ=1 second, Tₑ=3 seconds, RR=10 BPM, and I:E=1:1.

The software is initiated by a signal provided by a user or automated process at block S3202. At block S3204, the inhalation state of the apparatus begins, and at block S3204 valve 204 is configured by adjusting control part 205 to divert air from port 216 through port 214 and into pump 208, and valve 206 is configured to release air into the atmosphere via port 224.

At block S3208 a signal is sent to pump 208 to pulse and ramp up to a certain speed in order to increase air flow into conduit 150. At block S3210 the pressure sensor 434 is sampled, and at block S3212 the algorithm determines whether ITPᵢ has been reached within the set Tᵢ. If ITPᵢ has not been reached (as may be the case on the first ventilation cycle in particular), adjustments are made to the speed of pump 208 at block S3214 for the next ventilation cycle before proceeding to block S3216. If ITPᵢ has been reached, no adjustments are necessary, and the algorithm proceeds directly to block S3216. Blocks S3212 and S3214 may include PID calculations, as discussed above with respect to FIG. 26.

The exhalation state begins at block S3216. Following the beginning of the exhalation state, at block S3220, valve 204 is configured by adjusting control part 205 to intake air from port 218 and into pump 208, and valve 206 is configured by adjusting control part 205 to direct air through port 222 and into conduit 150. The rate at which control parts 205 are adjusted will depend on the set points inputted by the user. If ITPₑ<0 cmH₂O, no adjustment is made to control parts 205 if ITPₑ>0 cmH₂O and Tᵢ/Tₑ<2, then control parts 205 will switch as fast as possible as described above. If ITPₑ>0 cmH₂O and Tᵢ/Tₑ>2, then control parts 205 will move at a calculated rate (as above).

At block S3220 a signal is sent to reduce the speed of pump 208 to reduce air flow into conduit 150 to reach the ITPₑ setpoint at the desired Tₑ. At block S3222 the pressure sensor 434 is sampled, and at block S3224 the software determines whether ITPₑ has been reached at the Tₑ setpoint. If ITPₑ has not been reached (as may be the case on the first ventilation cycle in particular), adjustments are made to the speed of pump 208 at block S3226 for the next ventilation cycle before proceeding to block S3228. If ITPₑ has been reached, no adjustments are necessary and the algorithm proceeds directly to block S3228. Blocks S3224 and S3226 include PID calculations, as discussed above with respect to FIG. 26.

At block S3228 the software checks for user input (e.g., a change in set-points). At block S3230, it is determined whether the operation settings should be reconfigured, such as by loading a new configuration file. Reconfiguration may be required when a different mode of operation is desired. If the settings are not to be reconfigured, the software returns to block S3204 to repeat the inhalation state. If the settings are to be reconfigured, for example when starting a new operation mode, ventilation is stopped at block S3232.

In some embodiments, necessary adjustments to the apparatus at block S3226 may instead be made immediately prior to block S3204.

Second fluid system is also controlled by control system 200, but may operate autonomously from the first fluid system to provide a constant positive airway pressure (CPAP) to the lungs 130. Pump 506 will be operated at a specific speed to achieve and/or maintain a user defined CPAP setpoint, corresponding to a desired pressure in airway 170. Adjustment of pump 506 is based on feedback from sensor 522, which may be configured to detect the pressure within conduit 511. In the example shown in the respiratory waveform shown in FIG. 25, the pressure inside airway 170, as measured by sensor 552 may be 7.5 cmH₂O.

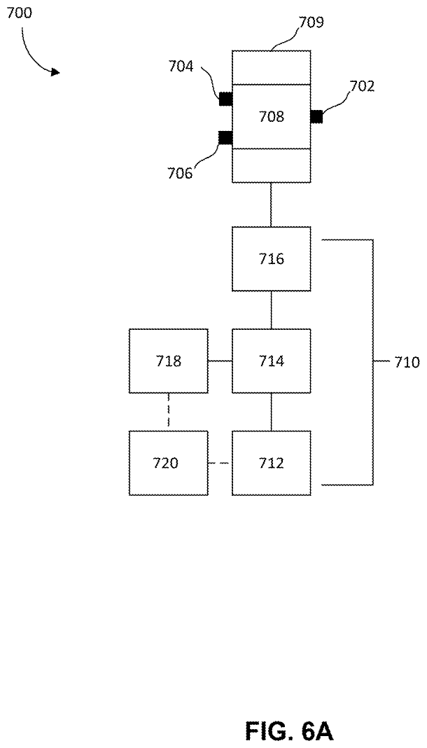

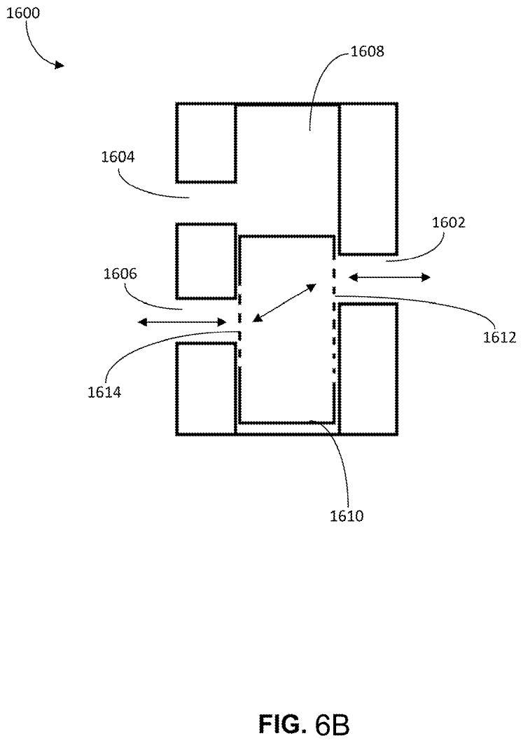

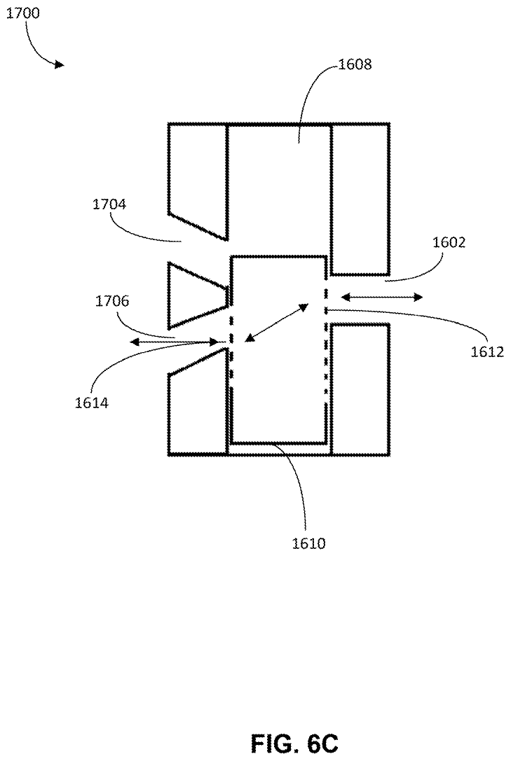

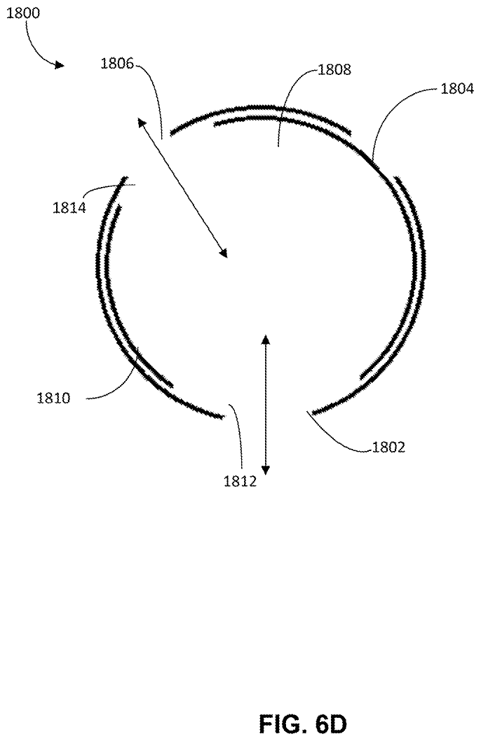

FIG. 6A is a schematic diagram illustrating additional components of a valve 700 suitable for use in any of the fluid systems 140, 160, 200 or 400 described above as a specific embodiment of valves 204 and 206. As described above, valve 700 has a port 702 grouped on one side of the valve that is in communication with a second group of ports 704 and 706 on the opposite side via conduit 709. To perform the function of control part 205, valve 700 has a spool 708, which is movable within conduit 709 in order to direct the flow of fluid between the two groups of ports. By doing this, valve 700 can selectively adjust a proportion of fluid flowing through ports 704 and 706 in order to adjust flow rates and pressures within any of the fluid systems described above without changing the pump speed. Spool 708 is moved by an actuator 710 between at least 2 positions. Actuator 710 comprises a motor 712, a driveshaft 714, a connector 716 and a proximity sensor 718. Driveshaft 714 is coupled to spool 708 by connector 716 and functions to position spool 708. Proximity sensor 718 determines the home position of driveshaft 714 and provides an output signal to a controller 720, which controls the position of driveshaft 714 through motor 712.

FIGS. 6B-D illustrate three generic embodiments of valve 700 in order to illustrate the use of a movable spool to control fluid flow between two groups of ports.

Referring to FIG. 6B, valve 1600 includes three ports in communication with cylindrical bore 1608; a port 1602 grouped on one side and ports 1604 and 1606 grouped together on the opposite side. In order to selectively control the flow between the two groups of ports, spool 1610 is housed within bore 1608. The spool is movable linearly within bore 1608 and is able to block or allow flow between the two groups of ports. In the position shown in FIG. 6B, flow though port 1604 is blocked whilst flow to/from port 1602 to/from port 1606 is possible through openings 1612 and 1614 in the spool (as indicated by the directional arrows). As will be described in greater detail below, the position of spool 1610 may be adjusted in order to precisely control the relative fluid flow to or from each of second and third ports 838 and 840.

Referring to FIG. 6C, a valve 1700 which is similar in design and function as valve 1600 is depicted. In this embodiment ports 1704 and 1706 are grouped together in a roughly V-shaped arrangement. One advantage of this configuration is that the pressure drop across the valve is lower due to the flow path exiting at 45 degrees, rather than 90 degrees. This will increase the efficiency of the valve when installed in a fluid system such as 300.

Referring to FIG. 6D, valve 1800 has three ports in communication with a cylindrical bore 1808, a port 1802 grouped on one side and ports 1804 and 1806 grouped together on the opposite side. In this embodiment, spool 1810 is housed within bore 1808 and is configured to move rotatably to block or allow flow between the two groups of ports. In the position shown in FIG. 6D, flow though port 1804 is blocked whilst flow to/from port 1802 to/from port 1806 is possible through openings 1812 and 1814 in the spool (as indicated by the directional arrows).

Of course, other positions for spools 1610, and 1810 are possible to open and close different pathways for fluid flow as will be outlined in more detail below.

A first embodiment of a valve 800 suitable for use in any of the fluid systems is shown in FIGS. 7A-B, 8A-B, 9A-C and 10A-F.

Referring in particular to FIGS. 7A-B and 8A-B, valve 800 has a generally rectangular valve base 802, to which a housing 804 is attached to the top surface 802a using a suitable method such as screws. Housing 804 has a rectangular cuboid shape, including a recessed spool opening 808 located on an end surface 804b of housing 804. Housing 804 may be made of a suitable material such as aluminum. A lid plate 810 is sized to fit within spool opening 808 and is secured in place using a suitable method such as screws 812. An O-ring 809, which is located within a groove 811 of spool opening 808 provides a seal between spool opening 808 and lid plate 810. O-ring 809 may be made of rubber or any other suitable material.

A motor mount 814 is positioned on the top surface 804a of housing 804. Motor mount 814 is generally flat and rectangular and provides a surface for mounting motor 816. In this embodiment, motor 816 may be a linear DC servomotor, such as a LM1247-C20-C1 linear DC brushless micromotor manufactured by MicroMo Electronics™. Motor 816 includes a driveshaft 820. Motor mount 814 also has a flanged tab 822 protruding perpendicular to the top surface 804a to which a proximity sensor 824 may be attached. Proximity sensor may be any suitable sensor, such as a GX-F8A-P inductive proximity sensor manufactured by Panasonic. As will be explained in further detail below, proximity switch is configured to detect the position of driveshaft 820, in particular to detect a home position for the driveshaft. Valve base 802, housing 804, lid plate 810 and motor mount 814 may be made from any suitably strong material such as aluminium.

Also secured to the top surface 802a of valve base 802 is an electronics mount 826. Electronics mount 826 comprises two feet 828 which contact the surface 802a of valve base 802 and are secured with a suitable method such as screws (not shown). Electronics mount 826 further includes two legs 830, extending vertically upwards from feet 828 to a vertical rectangular backing plate 832. A controller 834 is secured to backing plate 832. In this embodiment, controller 834 may be a MCLM3002SRS motor controller manufactured by MicroMo Electronics™ Electronics mount 826 may be made from any suitably strong material such as acetyl plastic.

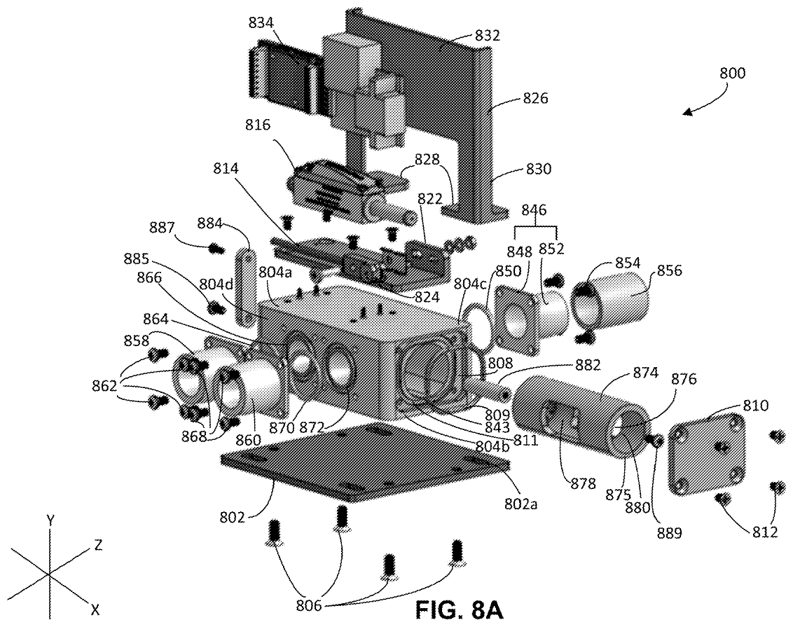

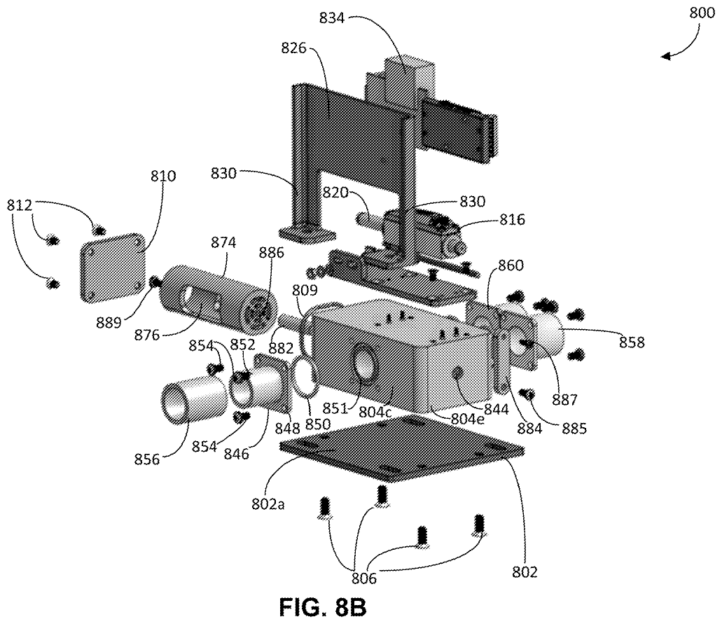

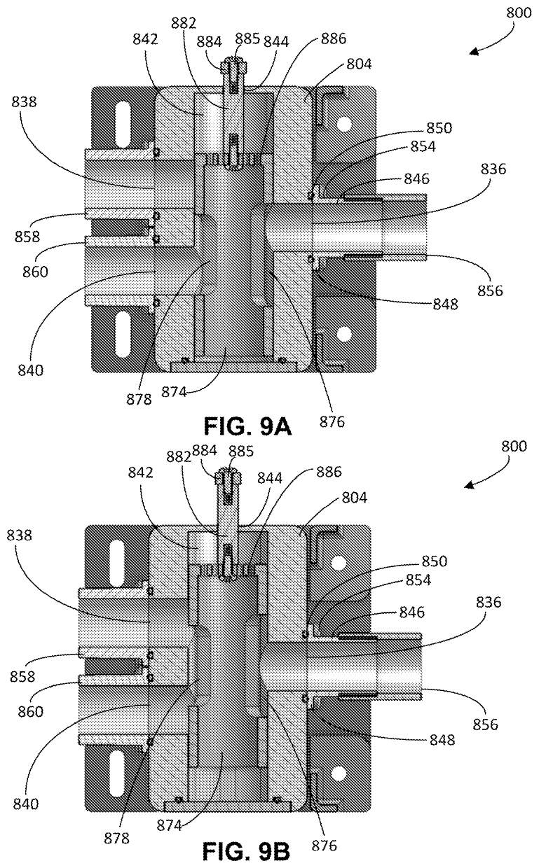

With reference to FIGS. 8A-B and FIGS. 9A-C in particular, housing 804 further includes a first port 836, a second port 838 and a third port 840. First port 836 is located on face 804c of housing 804, whilst second and third ports are located on the opposite face 804d of housing 804. Ports 836, 838 and 840 are cylindrical passageways that extend inwards into the center of housing 804 to communicate with a central cylindrical bore 842. Cylindrical bore 842 extends along the longitudinal axis of housing 804, terminating at one end at spool opening 808. At the opposite end of cylindrical bore 842 is rod connector opening 844, which exits through face 804e of housing 804 (FIG. 8B). Cylindrical bore 842 may not be completely circular in cross-section and may comprise a flat portion 843 located on the bottom surface.

In order to provide a fluid connection between first port 836 and any tubing or conduits used to either receive fluid from or supply fluid to valve 800, a first port connector 846 is provided comprising a flanged portion 848 and a tubular portion 852 (FIG. 8B). The flanged portion 848 of first port connector 846 contacts the surface 804c of housing 804 where first port 836 exits housing 804. In order to provide a seal, an O-ring 850 that is located in a groove 851, following the circumference of port 836 on surface 804c, may be provided to seal between housing 804 and first port connector 846 when secured by screws 854. The tubular portion 852 of first port connector 846 is sized to receive a tubing fixture or tubing of a conduit such as tubing 856 as shown in FIG. 8B. Tubing 856 may be secured by any suitable means, such as by a tubing clamp (not shown).

Similar to as described above, a second port connector 858 and a third port connector 860 are also provided to provide a fluid connection between respective second and third ports 838, 840 and any tubing or conduits used to either receive fluid from or supply fluid to valve 800. Second port connector 858 is mounted to face 804d of housing 804 using screws 862 and is sealed with O-ring 864, which is located in a circular groove 866 in face 804d. Similarly, third port connector 860 is mounted to face 804d of housing 804 using screws 868 and is sealed with O-ring 870, which is located in groove 872 in face 804d which follows the circumference of port 860. First, second and third port connectors 846, 858, 860 may be made from any suitably strong material such as aluminium. O-rings 850, 864, 870 may be rubber or any other suitable material.

In order to selectively adjust a proportion of fluid flow through the second and third ports 838, 840 a spool 874 is provided, which is mounted within cylindrical bore 842. Spool 874 is hollow and generally cylindrically shaped with three openings on the surface of the cylinder and is sized to snugly fit within cylindrical bore 842. The outer surface of spool 874 may include a flattened region 875 that aligns with flat portion 843 of cylindrical bore 842, which ensures that the spool is correctly aligned within the cylindrical bore during assembly (FIG. 8A). A first opening 876, generally rectangular in shape, is located on the curved surface of spool 874. A second, smaller generally rectangular opening 878 is located on the curved surface, opposite to first opening 876. A third opening 880 is located on the end of spool 874 proximal to lid plate 810. Spool 874 may be made from any suitably strong material such as acetal plastic.

Spool 874 may be sized such that the clearance between its outer surface and the cylindrical bore is about 0.15 mm. Spool 874 may be manufactured from a suitable material with a low coefficient of friction, such as acetyl plastic.

The closed end 886 of spool 874 may contain a series of perforations (FIG. 8B) that function to allow fluid to flow therethrough, preventing trapped fluid impeding movement during the actuation of spool 874. A central shaft 882, cylindrical with threaded holes at either end, connects spool 886 to connector 884, which is in turn connected to driveshaft 820 for moving the spool. Central shaft 882 is coupled to the closed end 886 of spool 874 by screw 889.

As will be explained below, spool 874 is movable between a number of positions through linear movement along the longitudinal axis of the cylindrical bore 842 to proportionally control fluid flows though valve 800. The actuating mechanism includes motor 816, proximity sensor 824 and a driveshaft 820 that connects spool 874 to motor 816. In this embodiment, driveshaft 820 comprises a cylinder with a threaded hole at one end coupled to the upper end connector 884 by a screw 887. The distal end of central shaft 882 of spool 874 is coupled to the lower end of connector 884 by a screw 885 that is received in the threaded end hole of central shaft 882.

Movement of spool 874 is controlled by motor 816. Motor 816 may be operable to move spool 874 in increments of 0.006 mm over a total stroke length of 17.3 mm. Activation of motor 816 (by controller 834) causes driveshaft 820 to move linearly along the x-axis indicated in FIG. 8A. This linear movement is translated to spool 874 through central shaft 882 and connector 884, resulting in movement of spool 874 along the same x-axis. Proximity sensor 824 functions to detect the home position of driveshaft 820 and produces an output signal for controller 834, allowing spool 874 to be positioned in any number of locations along the longitudinal axis of cylindrical bore 842 in order to control the flow of fluid through valve 800.

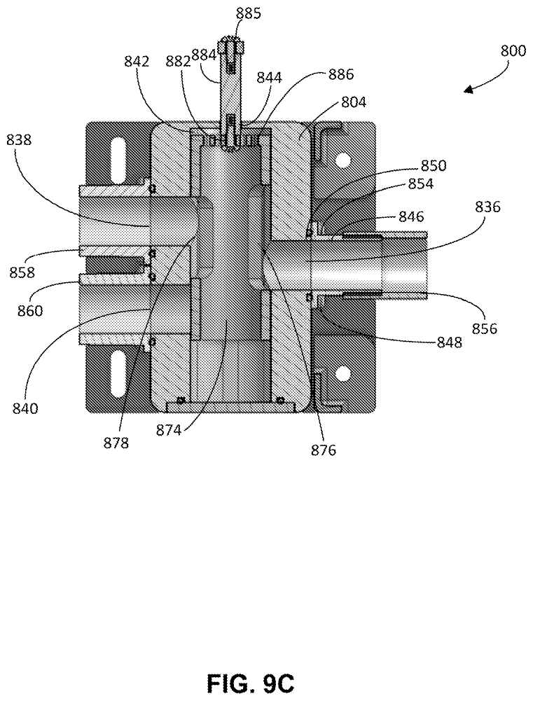

With reference to FIGS. 9A-C and 10A-F, three positions for spool 874 are illustrated. Turning first to FIGS. 9A and 10A-B, spool 874 is in a first position (also referred to a home position), with the open end of spool 874 adjacent to lid plate 810. In this position, first opening 876 is in alignment with first port 836 and second opening 878 is in alignment with third port 840. Depending on the configuration of the system valve 800 is installed in, fluid may be able to flow into third port 840, through second opening 878, through spool 874 and flow through first port 836 via first opening 876. Alternatively, fluid may be able to flow into first port 836, through first opening 876, through spool 874 and flow though third port 840 via second opening 878.

As described above, spool 874 may be actuated into a second position as shown in FIGS. 9B and 10C-D. In this position, first opening 876 is still in alignment with first port 836 and second opening 878 is in alignment with second port 838 and third port 840. Depending on the configuration of the system valve 800 is installed in, fluid may be able to flow into both or either second port 838 and third port 840, through second opening 878 into through spool 874 and flow out through first port 836 via first opening 876. Alternatively, fluid may be able to flow into first port 836, through first opening 876, through spool 874 and flow though second port 838 and third port 840 via second opening 878.

Spool 874 may be actuated into a third position as shown in FIGS. 9C and 10E-F. In this position, first opening 876 is still in alignment with first port 836 and second opening 878 is in alignment with second port 838. Depending on the configuration of the system valve 800 is installed in, fluid may be able to flow into second port 838, through second opening 878, through spool 874 and flow through first port 836 via first opening 876. Alternatively, fluid may be able to flow into first port 836, through first opening 876, through spool 874 and flow though second port 838 via second opening 878.

The position of spool 874 within cylindrical bore 842 may not be limited to the three positions illustrated in FIGS. 9A-C. Through control of the position of spool 874, the relative fluid flow to/from each of second and third ports 838 and 840 may be precisely controlled.

First opening 876 may be sized such that, regardless of the position of spool 874 within cylindrical bore 842 fluid flow is always possible between first port 836 and first opening 876. Second opening 878 may be sized such that, depending on the position of spool 874 within cylindrical bore 842, fluid flow is possible between second opening 878 and only second port 838, only third port 840 or second and third ports at the same time.

In some embodiments, spool 874 may have a length of 64.1 mm and a diameter of 24.8 mm. First opening 876 may have length of 32.6 mm and second opening 878 may have a length of 25.5 mm. First port 836, second port 838 and third port 840 may each have a diameter of 15.3 mm.

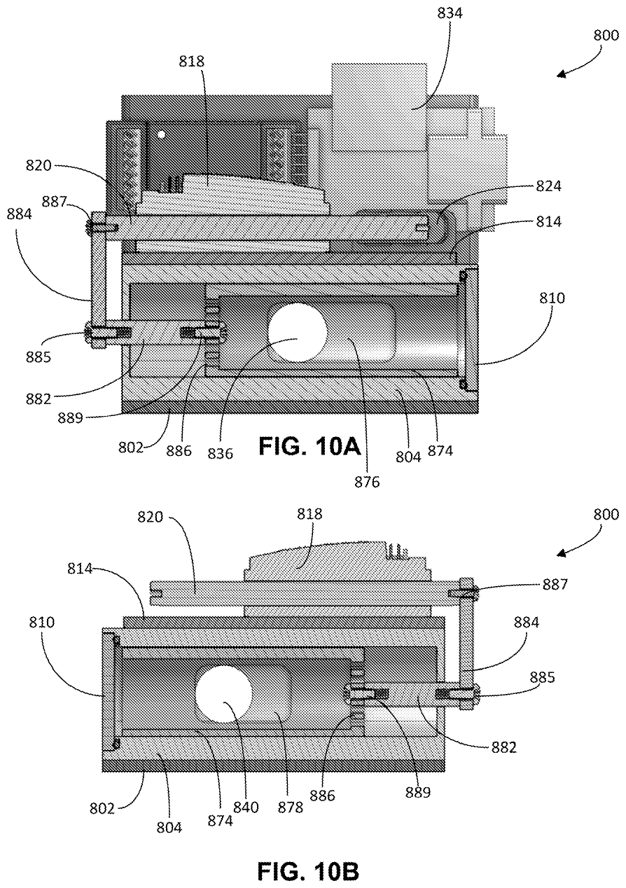

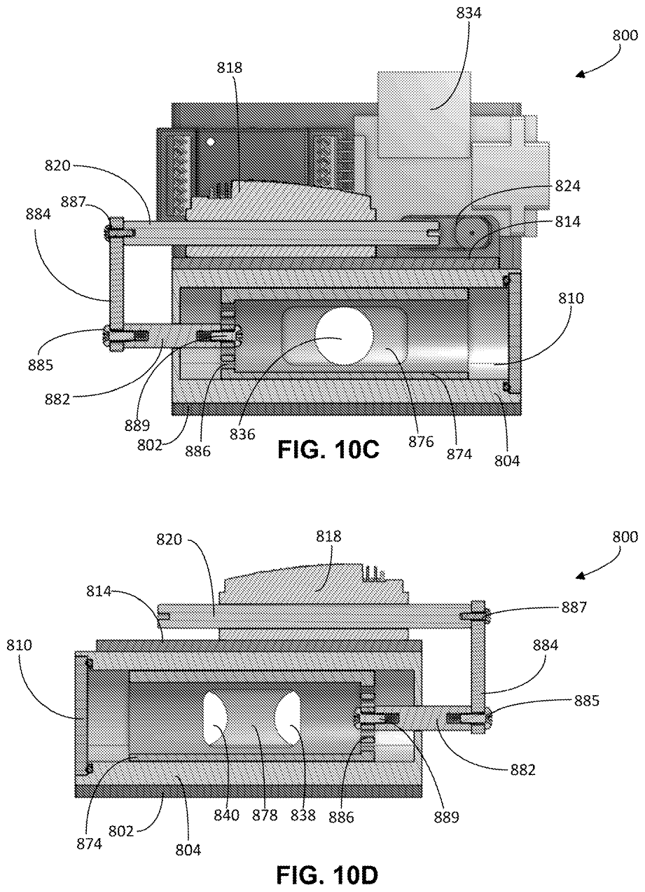

Turning to FIGS. 11A-B, 12A-B and 13A-B, another embodiment of a valve 900 is shown, suitable for use in any of the first and second fluid systems described above. Similar to valve 800, valve 900 includes valve base 802, housing 804, motor mount 814, motor 816 and proximity sensor 826. Mounted to the top surface 802a of valve base 802 is electronics mount 926. In this embodiment, electronics mount 926 comprises four feet 928 which contact the surface 902a at each corner of valve base 802 and are secured with a suitable method such as screws (not shown). Connected to each of the four feet 928 are four legs 930, extending vertically upwards from feet to backing plate 932. Backing plate 932 comprises two spaced apart parallel plates 932a and 932b. A controller 934 is secured to the top surface of backing plate 932a.

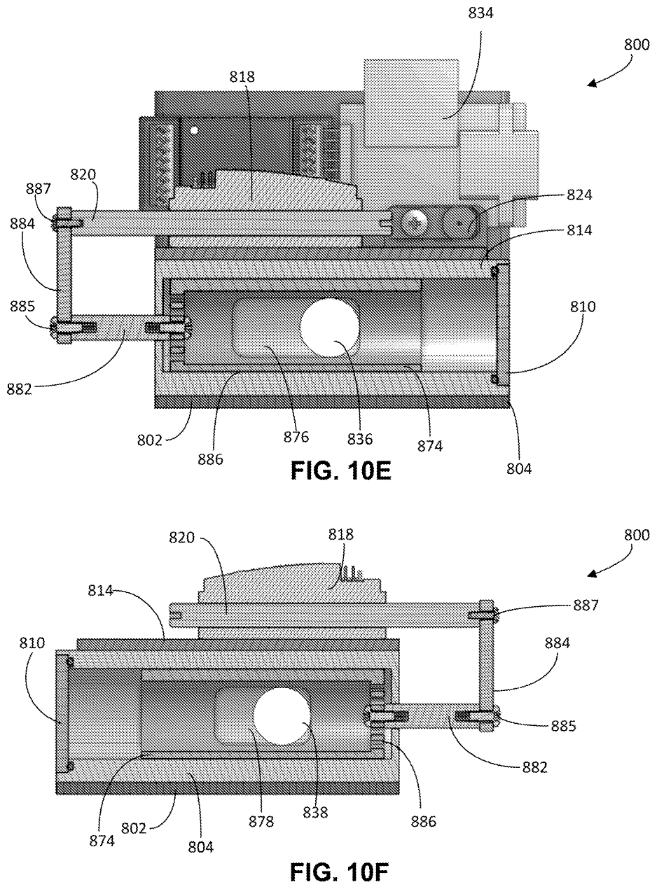

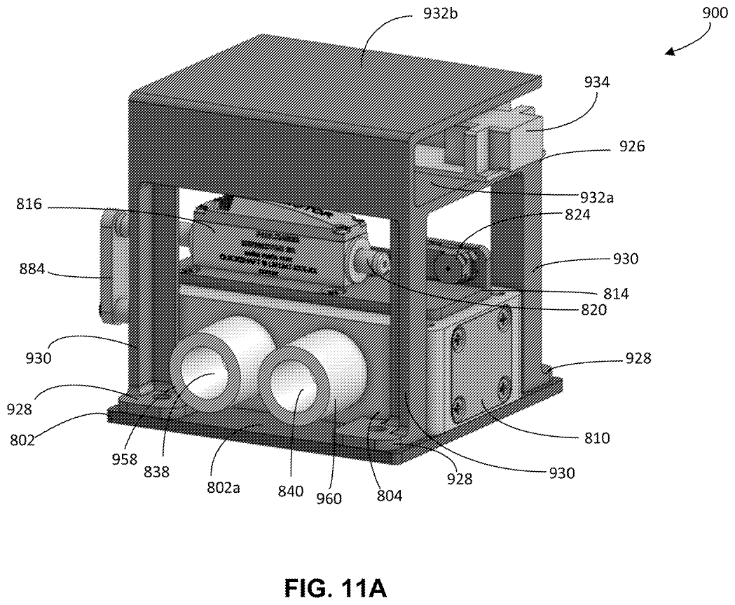

With reference to FIGS. 12A-B, housing 804 includes a first port 836, a second port 838 and a third port 840 that cooperate with cylindrical bore 842 as described above for valve 800. In order to provide a fluid connection between first port 836 and any tubing or conduits used to either receive fluid from or supply fluid to valve 800, a first port connector 946 is provided. First port connector 946 includes a first tubular portion 952 for receiving tubing or conduits and a second, narrower tubular portion 953 sized to fit within first port 836 of housing 804 by an interference fit.

Similarly, second port connector 958 and a third port connector 960 are also provided to provide a fluid connection between respective second and third ports 838, 840 and any tubing or conduits used to either receive fluid from or supply fluid to valve 900.

In order to selectively adjust a proportion of fluid flowing through the second and third ports 838, 840 a spool 874 is provided, that operates in a similar manner to as described above for valve 800.

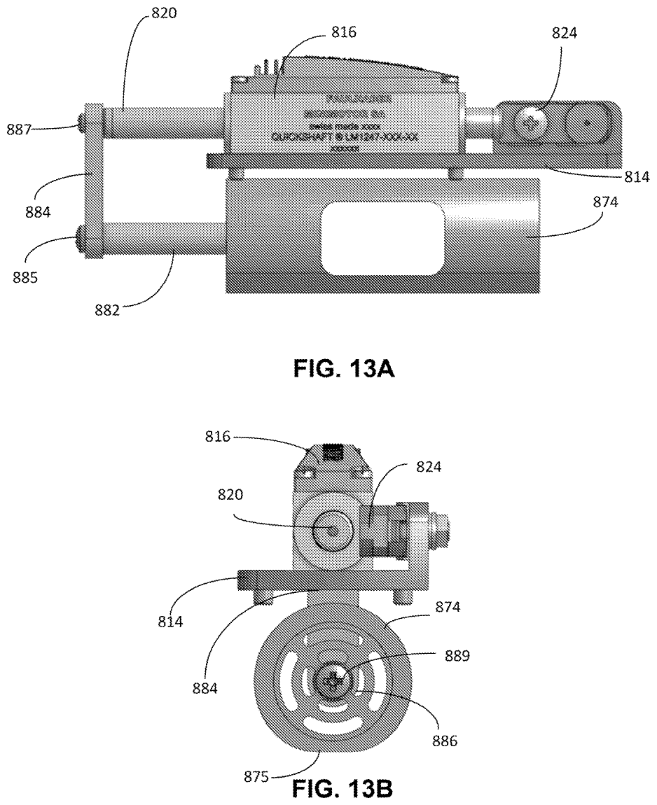

Another embodiment of a valve 1000 is shown in FIGS. 14, 15, and 16A-16D, suitable for use in any of the first and second fluid systems described above. Valve 1000 includes valve base 1002, housing 1004, motor mount 1014 and guide rail 1088.

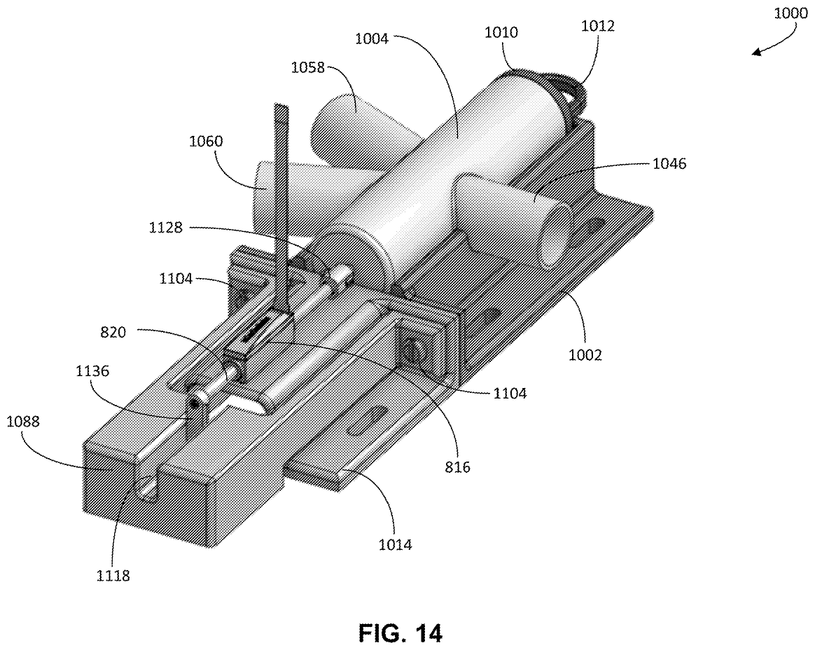

With particular reference to FIGS. 14 and 15, valve base 1002 has a rectangular base 1090, the proximal end of which terminates in flanged end 1092. Running longitudinally down the center of mounting base 1002 is a housing mount 1094, with a semi-cylindrical profile sized to receive a housing 1004. The semi-cylinder of housing mount 1094 is closed at the distal end and open at the proximal end.

Motor mount 1014 has an approximately inverted U-shape cross-sectional profile with a top surface 1096 and side surfaces 1098 and 1100, which run longitudinally down the center, terminating in a flanged end 1102. The outer face of flanged end 1098 contacts the outer face of flanged end 1092 of valve base 1002. A motor 816, orientated longitudinally and in alignment with housing 1004 is mounted to the top surface 1096 of motor mount 1014. In this embodiment motor 816 may be a linear DC servomotor, such as a LM0830-C15-C1 linear DC brushless micromotor manufactured by MicroMo Electronics™.

A guide rail 1088 is coupled to motor mount 1014. Guide rail 1088 comprises a roughly rectangular body 1108 at the distal end with first and second parallel arms 1110 and 1112 extending longitudinally (z-axis on FIG. 15) from body 1108, terminating in flanged brackets 1114 and 1116. When fitted to valve 1000, side surfaces 1098 and 1100 of motor mount 1014 are snugly sandwiched by first and second parallel arms 1110 and 1112, as shown in FIG. 14. Flanged brackets 1114 and 1116 contact the inner face of flanged end 1102 and guide rail 1098, motor mount 1014 and valve base 1002 are secured with a suitable method such as bolts 1104 and nuts 1106. Running longitudinally down the center of the top surface of rectangular body 1108 is a guide channel 1118, a recessed channel extending approximately half the depth of body 1108.

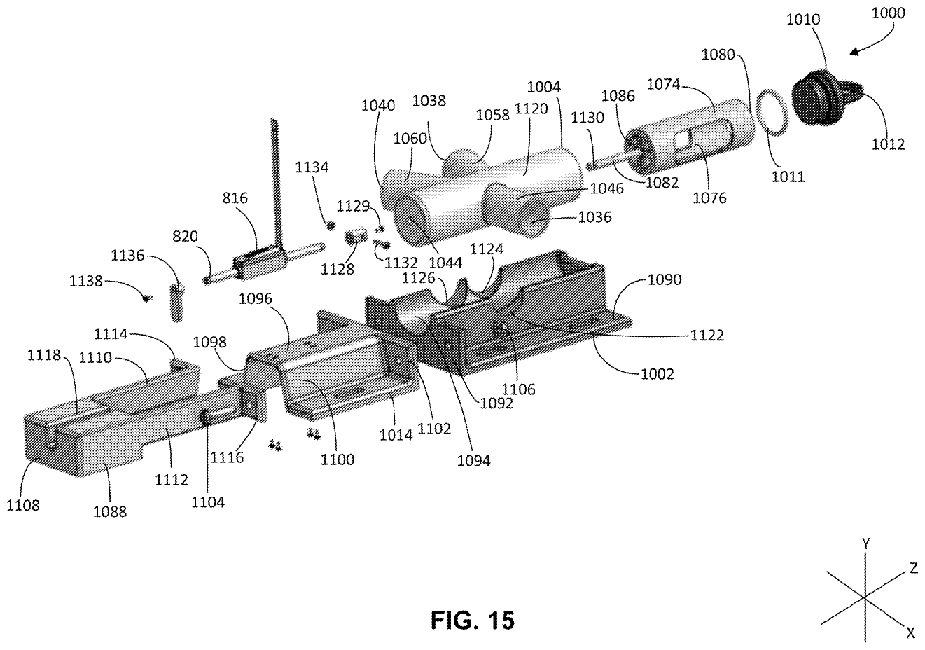

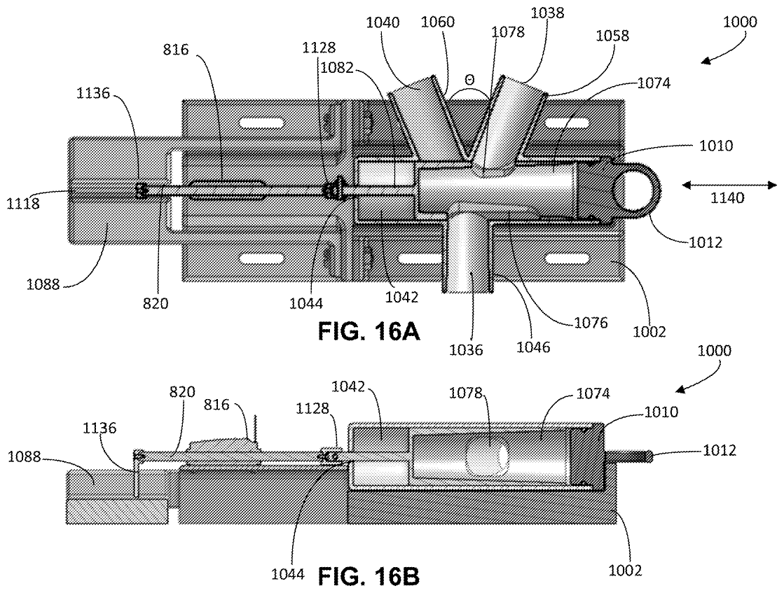

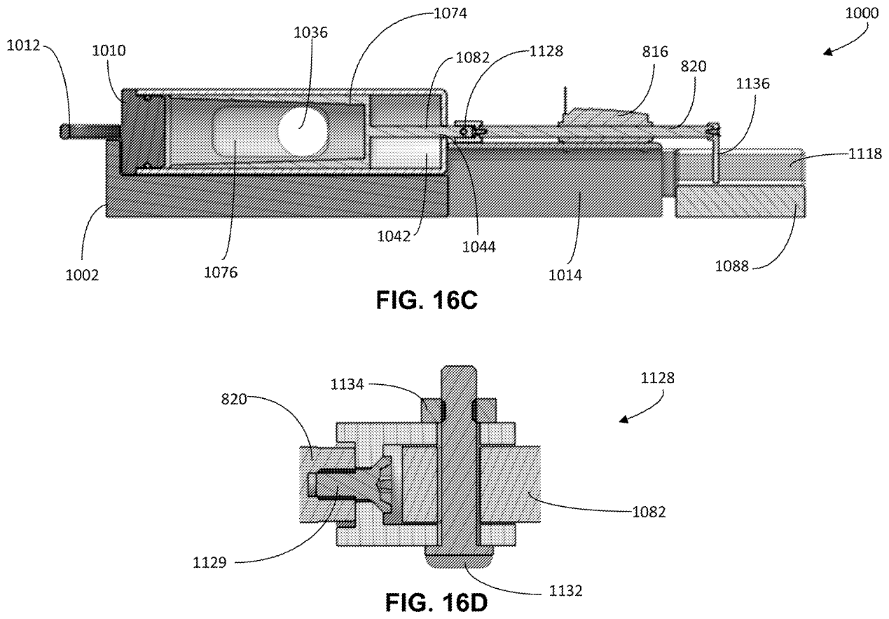

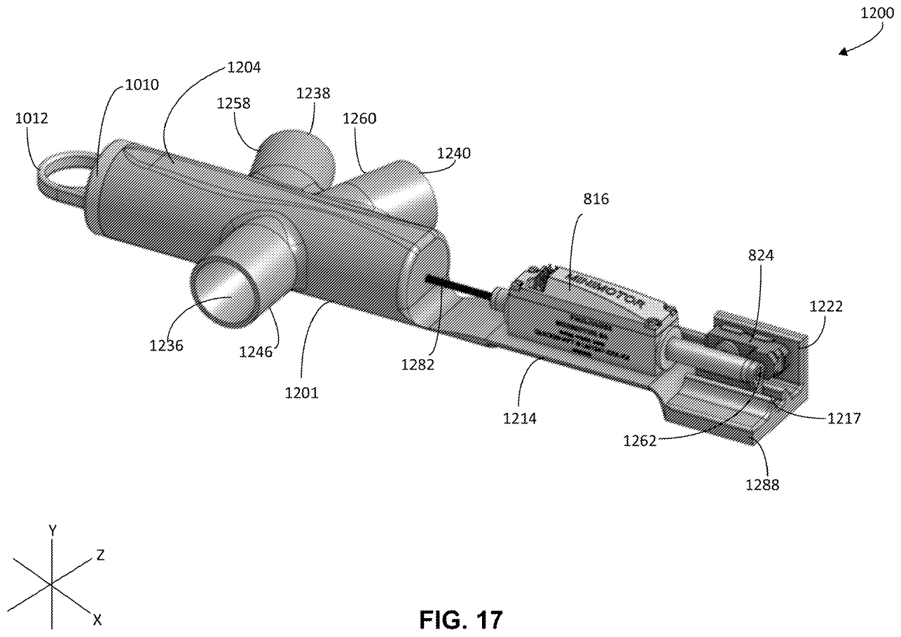

Mounted to housing mount 1094 of valve base 1002 is housing 1004. With reference to FIGS. 15 and 16A-D, housing 1004 has a cylindrical body 1120, open at the distal end with a cylindrical bore 1042 defined by the inner surface. The distal end is sealed with removable end cap 1010 which may comprise a ring 1012 protruding from the outer end to aid with removal. Housing 1004 may be made of a suitable material such as aluminum. An O-ring 1011 (FIG. 15) provides a seal between cylindrical body 1120 and end cap 1010. O-ring 1011 may be any suitable material such as rubber. Cylindrical body 1120 has a first port 1036 extending perpendicularly outwards from cylindrical body 1120, located at approximately the mid-point along the length of body 1120. Housing 1004 further also includes a second port 1038 and a spaced apart third port 1040 on the opposite side of the cylindrical body 1120 to first port 1036. Housing 1004 also includes a central shaft opening 1044 at the proximal end (FIG. 15).

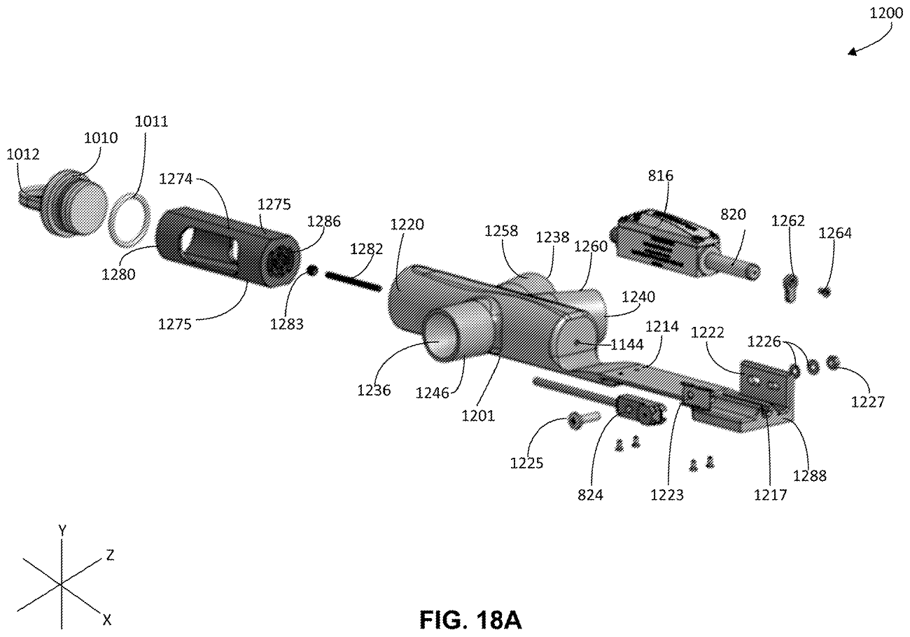

In order to provide fluid communication between first, second and third ports 1036, 1038, 1040 and any tubing or conduits used to either receive fluid from or supply fluid to valve 1000, first, second and third port connectors 1046, 1058 and 1060 are formed as an integral part of housing 1004. Second and third port connectors 1058, 1060 are angled away from each other in order to reduce the pressure drop across the valve whilst providing sufficient clearance between them, for attaching tubing or conduits. In one embodiment, the angle θ between second and third port connectors 1058, 1060 in FIG. 17A is between 43.5-44.5 degrees and optimally 44 degrees.

The lower half of cylindrical body 1120 of housing 1004 is sized to fit within housing mount 1094 with first, second and third port connectors 1046, 1058 and 1060 positioned such that they are located within respective first, second and third cutouts 1122, 1124, 1126 of housing mount 1094 (FIG. 15).

Similar to other valve embodiments, in order to selectively adjust a proportion of fluid flowing through second and third ports 1038, 1040 a spool 1074 is provided, mounted within cylindrical bore 1042. Similar to spool 874, spool 1074 is hollow and cylindrically shaped with three openings on the surface of the cylinder and is sized to snugly fit within cylindrical bore 1042. A first opening 1076, generally rectangular in shape is located on the curved surface of spool 1074. A second, smaller generally rectangular opening 1078 is located on the curved surface, opposite to first opening 1076. A third opening 1080 is located on the end of spool 1074 proximal to end cap 1010. Similar to spool 874, the closed end 1086 of spool 1074 may contain a series of perforations. A central shaft 1082 comprising a cylinder with a threaded hole 1130 at the distal end protrudes from the center of the closed end 1086. In this embodiment, central shaft 1082 and spool 1074 are a single unitary piece. When installed within housing 1004, central shaft 1082 protrudes through opening central shaft opening 1044 (FIG. 15).

Similar to spool 874 in valve 800, spool 1074 is moveable between a number of positions to control fluid flow through valve 1000 in substantially the same manner as described above (similar to as shown in FIGS. 9A-C). The actuating mechanism for valve 1000 comprises motor 816, driveshaft 820 that couples spool 1074 to motor 816 via connector 1128. In this embodiment, the driveshaft 820 and spool 1074 are in axial alignment along the z-axis indicated in FIG. 15. Shown in more detail in FIG. 16D, connector 1128 has a cylindrical body with openings at either end to receive central shaft 1082 and driveshaft 820. Driveshaft 820 is secured with screw 1129, which is received within a threaded opening within the driveshaft. To secure spool 1074 to connector 1128, connector 1128 may also comprise a hole positioned along its length that aligns with a hole 1130 in the distal end of central shaft 1082 for locating a bolt 1132 therethrough. Bolt 1132 is secured with nut 1134.

At the distal end of driveshaft 820, a guide rod 1136 is secured by screw 1138. Guide rod 1136 is generally rectangular with rounded ends and extends downwards to be received by guide channel 1118. Guide rod 1136 is sized to be located in guide channel 1118 during the full range actuation of valve 1000 and serves to keep driveshaft 820, connector 1128, central shaft 1082 and spool 1074 in alignment during actuation.

Movement of spool 1074 is controlled by motor 816. Activation by a controller (not shown) causes driveshaft 820 to move linearly in the directions indicated by arrow 1140 in FIG. 16A. This linear movement is translated to spool 1074 through central shaft 1082 via connector 1128, resulting in linear movement of spool 1074.

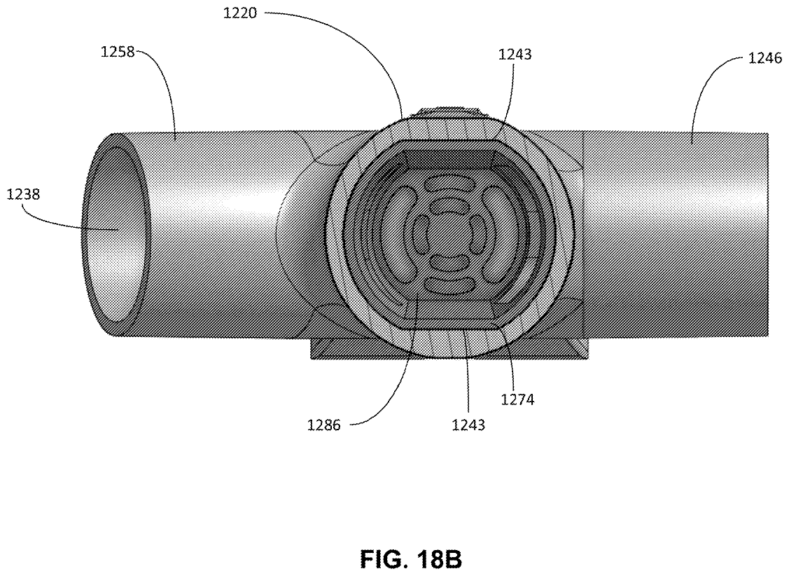

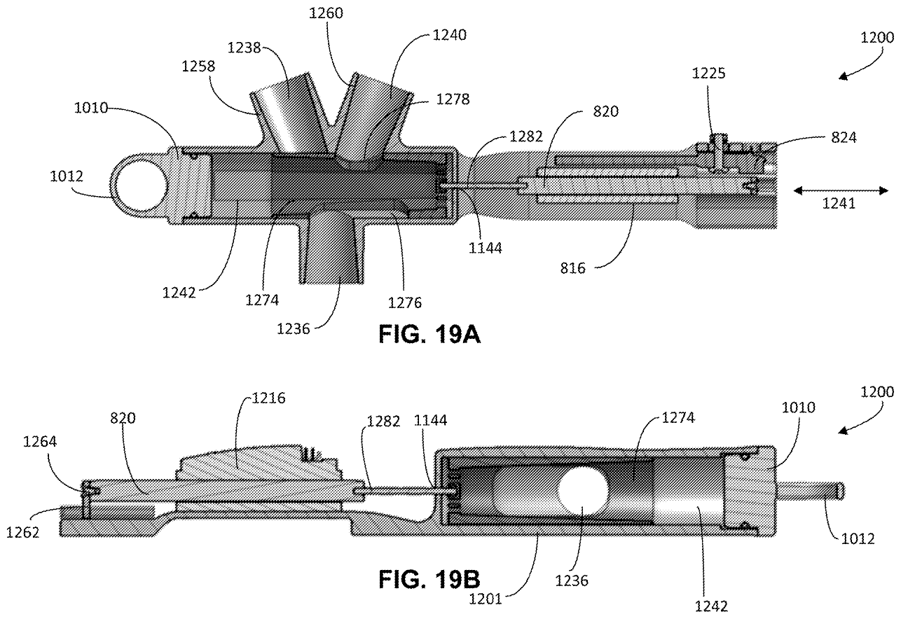

Another embodiment of a valve 1200 is shown in FIGS. 17, 18A-B and 19A-B, similar to valve 1000 and suitable for use in any of the first and second fluid systems described above. With reference to FIG. 17, valve 1200 includes a housing 1204, motor mount 1214 and guide rail 1288 formed as a single continuous valve body 1201. Motor mount 1214 is formed as a single, rectangular piece between the proximal ends of housing 1204 and guide rail 1288. Motor mount 1214 has a flat upper surface for receiving a motor 816.

Formed at the distal end of motor mount 1214, valve body 1201 further comprises a guide rail 1288 coupled to motor mount 1214. Guide rail 1288 comprises a guide channel 1217 extending the whole length longitudinally (x-axis on FIG. 17) down the center of guide rail 1288. Guide rail 1288 has a flanged tab 1222 protruding perpendicular to the top surface of guide rail 1288 to which proximity sensor 824 may be attached using mounting bracket 1223, bolt 1225, washers 1226 and nut 1227. Proximity sensor 824 is configured to detect the position of driveshaft 820.

Similar to housing 1004 of valve 1000 described above, housing 1204 has a cylindrical body 1220, open at the distal end with a cylindrical bore 1242 defined by the inner surface. In this embodiment, cylindrical bore 1242 has a circular cross section that has flat portions 1243 located at the top and the bottom (FIG. 18B). The distal end is sealed with removable end cap 1010 and O-ring 1011. Cylindrical body 1220 has a first port 1236 extending perpendicularly outwards from cylindrical body 1220, located at approximately the mid-point of the length of body 1220. Housing 1204 further comprises a second port 1238 and a spaced apart third port 1240 on the opposite side of the cylindrical body 1220 to first port 1236. Housing 1004 also includes a central shaft opening 1144 at the proximal end (FIG. 18A).

In order to provide fluid communication between first, second and third ports 1236, 1238, 1240 and any tubing or conduits used to either receive fluid from or supply fluid to valve 1200, first, second and third port connectors 1246, 1258 and 1260 are provided which are arranged and function in a similar manner to first, second and third port connectors 1046, 1058 and 1060 of valve 1000 described above.

Similar to other valve embodiments described above, in order to selectively adjust a proportion of fluid flowing through the second and third ports 1238, 1240 a spool 1274 is provided, mounted within cylindrical bore 1242. Similar to spool 1074, spool 1274 is hollow and cylindrically shaped with three openings on the surface and is sized to snugly fit within cylindrical bore 1242. A central shaft 1282 comprising a cylinder threaded at each end is coupled to the closed end 1286 of spool 1274 using heat set threaded insert 1283. In this embodiment, the top and bottom surfaces of spool 1274 comprise flattened portions 1275, sized to complement the flattened regions 1243 of cylindrical bore 1242 to ensure that the spool is correctly aligned within the cylindrical bore during assembly (FIG. 18A). This arrangement eliminates any rotation of spool 1274 within the cylindrical bore during operation, preventing central shaft 1282 unthreading from either the spool 1274 or driveshaft 820. A first opening 1276, generally rectangular in shape, is located on the curved surface of spool 1274. A second, smaller generally rectangular opening 1278 is located on the curved surface, opposite to first opening 1276. A third opening 1280 is located on the end of spool 1274 proximal to end cap 1010. Similar to spool 874, the closed end 1286 of spool 1274 may contain a series of perforations.

Similar to spool 874 in valve 800, spool 1274 is movable between a number of positions (similar to those shown in FIGS. 9A-C) in substantially the same manner as described above to control fluid flow through valve 1200. The actuating mechanism for valve 1200 includes motor 816 and driveshaft 820 that couples spool 1274 to motor 816. In this embodiment, the driveshaft 820 and spool 1274 are in axial alignment along the z-axis indicated in FIG. 18A. The distal threaded end of central shaft 1282 is coupled to the threaded center of driveshaft 820.

The actuating mechanism for valve 1200 may also include a proximity sensor 824 which functions to detect the position of driveshaft 820 (and consequently spool 1274) and produces an output signal for to the controller (not shown), allowing spool 1274 to be positioned in any number of locations along the longitudinal axis of cylindrical bore 1242 in order to control the flow of fluid through valve 1200.

At the distal end of driveshaft 820 a guide rod 1262is secured by screw 1264. Guide rod 1262 extends downwards into guide channel 1217. Guide rod 1262 is sized to locate in guide channel 1217 during the full range of actuation of valve 1200 and serves to keep driveshaft 820, central shaft 1282 and spool 1274 in alignment during actuation.

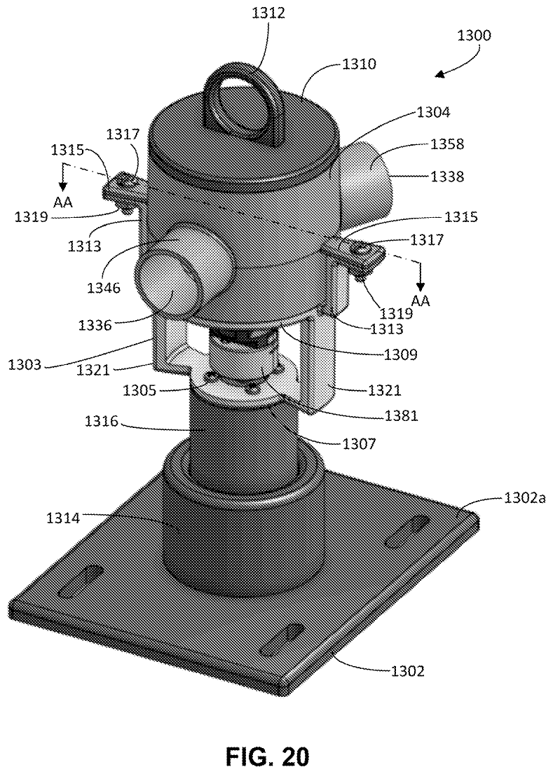

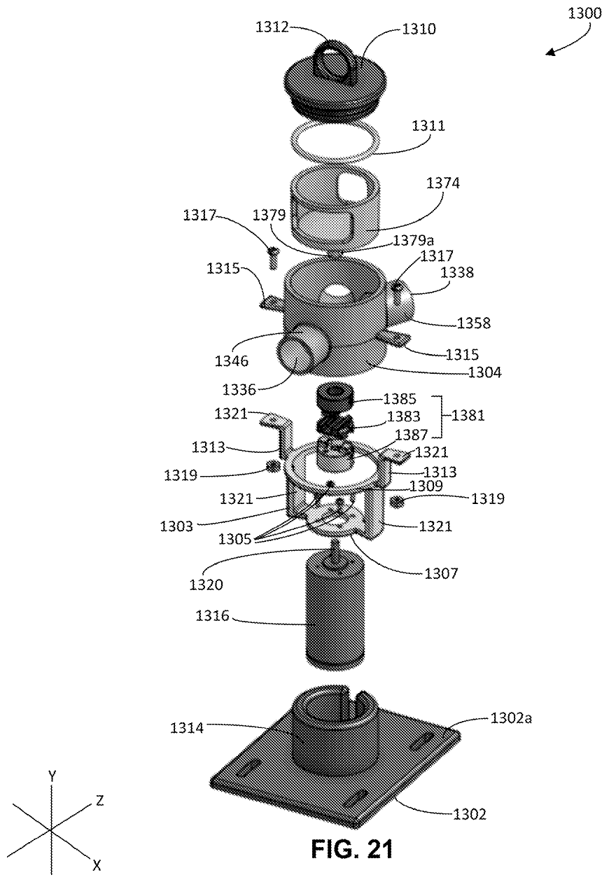

Movement of spool 1274 is controlled by motor 816. Activation of motor by the controller (not shown) causes driveshaft 820 to move linearly in the directions indicated by arrow 1241 indicated in FIG. 19A. This linear movement is translated to spool 1274 through central shaft 1282 resulting in linear movement of spool 1274.

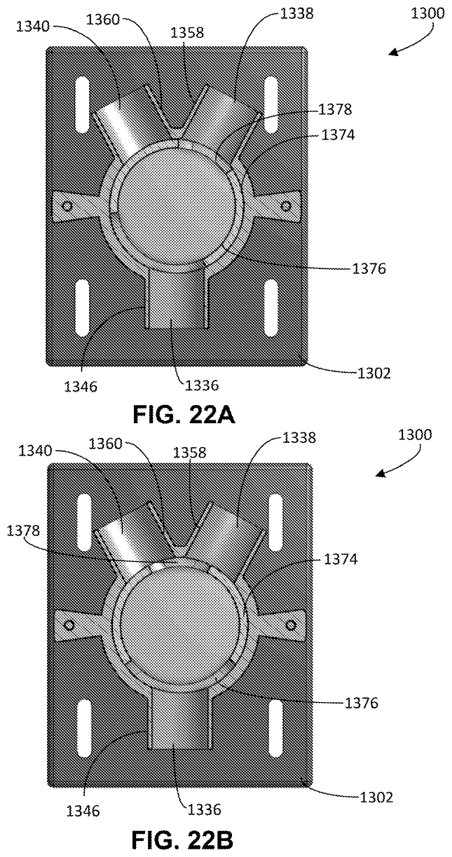

With reference to FIGS. 20, 21, 22A-C, 23 and 24, another embodiment of a valve 1300 suitable for use in any of the first or second fluid systems described above is depicted, which includes a valve base 1302, motor 1316, housing support 1303, housing 1304 and lid 1310.

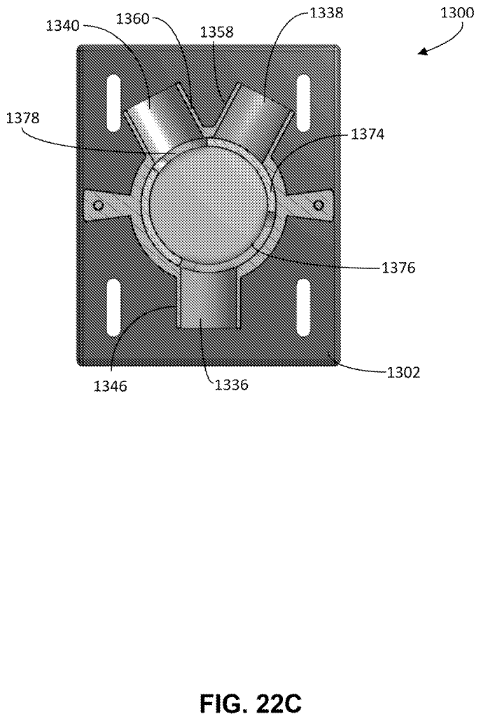

Referring in particular to FIGS. 20 and 21, valve base 1302 may include a rectangular plate 1302a and a motor mount 1314. Motor mount 1314 is a hollow cylinder protruding from the center of rectangular plate 1302a that is open at the upper end for receiving a motor 1326 therein. In this embodiment, motor 1316 may be a brushless DC-servomotor.

With refence to FIG. 21, housing support 1303 is coupled to motor 1316 and is secured by screws 1305 which are located into threaded holes on the top surface of motor 1316. Housing support 1302 comprises a lower housing mounting plate 1307 and an upper housing mounting plate 1309, linked by a pair of upwardly extending lower support arms 1321. Lower housing mounting plate 1307 is a round plate with an opening in the center for the actuation mechanism to fit therethrough. Similarly, upper housing mounting plate 1309 has a generally circular shape, sized to follow the outer profile of housing 1304, with a central opening. At opposite points along the circumference of upper housing mounting plate 1309 are two upper support arms 1313, rising vertically with flanged ends 1321 projecting horizontally outwards that contact corresponding flanges 1315 on housing 1304 to provide additional support. Flanged ends 1315 and flanges 1315 are secured together using bolts 1317 and nuts 1319.

In this embodiment, housing 1304 may be cylindrically shaped, with an opening at the upper end which may be reversibly sealed by lid 1310 and O-ring 1311. Lid 1310 may comprise a ring 1312 protruding from the upper surface to aid with removal. Housing 1304 further comprises a first port 1336, a second port 1338 and a third port 1340 positioned on the outer cylindrical surface. Second and third ports 1338, 1340 are positioned adjacent to each other whilst first port 1336 is positioned on the opposite side of housing 1304.

In order to provide a fluid connection between first port 1336, second port 1338 and third port 1340 and any tubing or conduits used to either receive fluid from or supply fluid to valve 1300, respective first, second and third port connectors 1346, 1358 and 1360 are provided that are formed as an integral part of housing 1304.

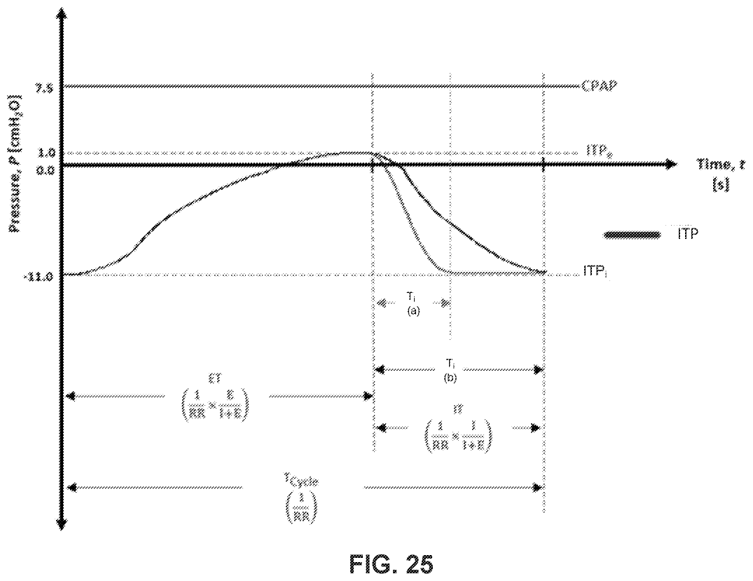

The inner surface of housing 1304 defines a cylindrical bore 1342 through which fluid may flow between first, second and third ports 1336, 1338 and 1340. In order to selectively adjust a proportion of fluid flowing through the second and third ports 1338, 1340 a spool 1374 is provided, sized to fit within cylindrical bore 1342 and shown in greater detail in FIG. 24. Spool 1374 has a hollow cylindrical body, closed at one end 1386 with a first rectangular opening 1376 on the curved surface and a second, smaller rectangular opening 1378 located on the opposite curved surface of spool 1374. A third opening 1380 is located at the top end of the cylinder of spool 1374. At the center of the bottom surface of the closed end 1386, protrudes actuation mount 1379, which provides a connection point for the actuation mechanism of valve 1300.

Spool 1374 is moveable between a number of positions in order to selectively adjust a proportion of fluid flow between first, second and third ports 1336, 1338 and 1340, through rotary movement about the central vertical axis of the cylindrical bore 1342 (y-axis in FIG. 21). The actuating mechanism comprises motor 1316 and driveshaft 1320, which are coupled using a connector. In this embodiment, in order to provide a secure connection with no backlash between spool 1374 and motor 1316, the connector is Oldham coupling 1381, which includes a disk 1383 sandwiched by upper and lower coupling hubs 1385 and 1387. An example of an Oldham coupling suitable of use in valve 1300 are MOST19-8-A and MOCT19-4-A coupling hubs and an OD12/19-AT coupling disk manufactured by Rotoprecision Inc. Upper coupling hub 1385 is attached to drive mount 1379 via a set screw (not shown) on the flat edge 1379a of drive mount 1379. Lower coupling hub 1387 is attached via an integrated clamping mechanism (not shown),

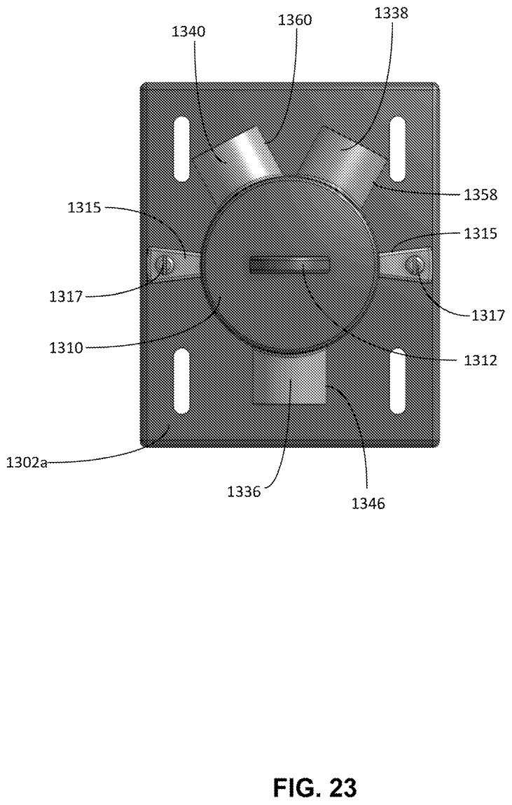

Movement of spool 1374 is controlled by motor 1316 which drives the rotation of spool 1374 via Oldham coupling 1381 about their central axes. With reference to FIGS. 22A-C, three positions for spool 1374 are illustrated. Turning first to FIG. 22A, spool 1374 is in a first position, with first opening 1376 in alignment with first port 1336 and second opening 1378 in alignment with second port 1338. Depending on the configuration of the system valve 1300 is installed in, fluid may be able to flow into second port 1338, through second opening 1378, through spool 1374 and flow through first port 1336 via first opening 1376. Alternatively, fluid may be able to flow into first port 1336, through first opening 1376, through spool 1374 and flow though second port 1338 via second opening 1378.

As described above, spool 1374 may be actuated into a second position as shown in FIG. 22B. In this position, first opening 1376 is in alignment with first port 1336 and second opening 1378 is in alignment with second port 1338 and third port 1340. Depending on the configuration of the system valve 1300 is installed in, fluid may be able to flow into second port 1338 and third port 1340, through second opening 1378, through spool 1374 and flow through first port 1336 via first opening 1376. Alternatively, fluid may be able to flow into first port 1336, through first opening 1376, through spool 1374 and flow though second port 1338 and third port 1340 via second opening 1378.

As described above, spool 1374 may be actuated into a third position as shown in FIG. 22C. In this position, first opening 1376 is in alignment with first port 1336 and second opening 1378 is in alignment with third port 1340. Depending on the configuration of the system valve 1300 is installed in, fluid may be able to flow into third port 1340, through second opening 1378, through spool 1374 and flow through first port 1336 via first opening 1376. Alternatively, fluid may be able to flow into first port 1336, through first opening 1376, through spool 1374 and flow though third port 1340 via second opening 1378.

When introducing elements of the present invention or the embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

Of course, the above-described embodiments are intended to be illustrative only and in no way limiting. The described embodiments of carrying out the invention are susceptible to many modifications of form, arrangement of parts, details, and order of operation. The invention, therefore, is intended to encompass all such modifications within its scope.

1. A valve assembly comprising:
a first conduit comprising a first end and a second end; and
a first control valve and a second control valve, each control valve comprising a first port, a second port, and a third port, and each control valve further comprising a spool configured to selectively adjust a proportion of fluid flows through the second port and the third port of the respective control valve,
wherein the first port of the first control valve is configured to connect to an intake port of a pump, the second port of the first control valve is configured to connect to the second end of the first conduit for withdrawing fluid from the first conduit, the first port of the second control valve is configured to connect to an output port of the pump, and the second port of the second control valve is configured to connect to the second end of the first conduit for supplying fluid to the first conduit.

2. The valve assembly of claim 1, wherein each control valve comprises an actuator for actuating the spool of the respective control valve, and wherein the actuator of each control valve comprises a servomotor.

3. (canceled)

4. The valve assembly of claim 2, wherein the spool of at least one each control valve of the first control valve and a second control valve is a sliding spool, and the actuator of the at least one control valve is a linear actuator.

5. The valve assembly of claim 2, wherein the spool of at least one control valve of the first control valve and a second control valve is a rotary spool, and the actuator of the at least one control valve is a rotary actuator.

6. The valve assembly of claim 4, wherein the linear actuator of the at least one control valve comprises a drive shaft coupled to the sliding spool of the respective control valve for positioning the sliding spool.

7. The valve assembly of claim 6, wherein the linear actuator of the at least one control valve comprises a proximity sensor for determining a position of the drive shaft of the respective control valve.

8. The valve assembly of claim 7, wherein the actuator of the at least one control valve comprises a controller for controlling movement of the drive shaft of the respective control valve based on an output signal from the proximity sensor of the respective control valve.

9. The valve assembly of claim 6, wherein the drive shaft of the linear actuator of the at least one control valve and the sliding spool of the respective control valve are axially aligned along an axis of the drive shaft.

10. The valve assembly of claim 9, wherein the driveshaft of the linear actuator of the at least one control valve comprises a guide rod configured to maintain the axial alignment of the drive shaft of the respective control valve.

11. The valve assembly of claim 6, wherein the linear actuator of the at least one control valve comprises a connector coupled to the drive shaft and the sliding spool of the respective control valve.

12. The valve assembly of claim 11, wherein the drive shaft of the linear actuator of the at least one control valve and the sliding spool of the respective control valve are vertically stacked.

13. The valve assembly of claim 1, wherein the at least one control valve comprises a housing including opposite ends and a cylindrical bore extending between the opposite ends, the first port of the respective control valve located at a first side of the bore and the second port of the respective control valve and the third port of the respective control valve located at a second side of the bore, and wherein the spool of the respective control valve is slidable in the bore and comprises a laterally extending conduit including a first opening facing the first side of the bore and a second opening facing the second side of the bore, the first opening sized and positioned to allow fluid communication with the first port, and the second opening sized and positioned to selectively allow fluid communication with the second port and the third port by sliding the spool in the bore.

14. The valve assembly of claim 1, comprising a three-way connection at the second end of the first conduit for connecting the second end of the first conduit to the second port of the first control valve and the second port of the second control valve respectively,
wherein the three-way connection comprises a three-way valve, a T-junction, or a Y-junction.

15. (canceled)

16. A ventilator comprising:
a sealed chamber for housing a lung in the sealed chamber, the sealed chamber comprising a pressure port; and
a first fluid system for applying a variable first pressure to an exterior surface of the lung in the sealed chamber through the pressure port to cause the lung to breathe, said first fluid system comprising:
a first pump comprising an intake port and an output port,
the valve assembly of claim 1 connected to the intake port and the output port of the first pump, wherein the first end of the first conduit of the valve assembly is sealingly coupled to the pressure port of the sealed chamber.

17. The ventilator of claim 16, further comprising a second fluid system for applying and maintaining a second pressure in an airway of the lung, wherein the second fluid system comprises a second conduit having a first end and a second end, the second end of the second conduit being connectable to a trachea of the lung through the sealed chamber.

18. The ventilator of claim 17, wherein the second fluid system further comprises a second pump for supplying a pressured fluid to the second conduit and maintaining the second pressure in the second conduit.

19. The ventilator of claim 17, further comprising a control system configured and connected for controlling the first fluid system and the second fluid system to cause the lung to breathe,
wherein the control system comprises sensors for sensing pressures and fluid flow rates in the first conduit and the second conduit, and a processor for processing the sensed pressures and fluid flow rates and determining a pumping speed of each pump and the proportions of fluid flows through the second port and the third port of the respective control valves.

20. (canceled)

21. The ventilator of claim 17, further comprising a third conduit connecting the third port of the second control valve of the first fluid system to the second conduit of the second fluid system, and a third control valve in the third conduit for regulating fluid flow from the first control valve to the second conduit through the third conduit.

22. The ventilator of claim 18, wherein each of the first pump and the second pump comprises a blower.

23. The ventilator of claim 16, comprising at least one of:
(a) a fluid filter in each of the first conduit and the second conduit;
(b) a fluid filter coupled to the third port of at least one of the first control valve and the second control valve; or
(c) a fluid filter coupled to an input port of the second pump.

24. (canceled)

25. (cancele
